SEQUENCE LISTING (2 sequences)

SEQ ID NO: 1 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 2 - DNA, 1940 bp, Homo sapiens
gccgcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaagcacaacagccaaaaaggactttccgctagacccactcgaggaaaactaaaaccttgtgagagatgaaagggcaaagacgtgggggagggggccttaaccatgaggaccaggtgtgtgtgtggggtgggcacattgatctgggatcgggcctgaggtttgccagcatttagaccctgcatttatagcatacggtatgatattgcagcttatattcatccatgccctgtacctgtgcacgttggaacttttattactggggtttttctaagaaagaaattgtattatcaacagcattttcaagcagttagttccttcatgatcatcacaatcatcatcattctcattctcattttttaaatcaacgagtacttcaagatctgaatttggcttgtttggagcatctcctctgctcccctggggagtctgggcacagtcaggtggtggcttaacagggagctggaaaaagtgtcctttcttcagacactgaggctcccgcagcagcgcccctcccaagaggaaggcctctgtggcactcagataccgactggggctgggcgccgccactgccttcacctcctctttcaacctcagtgattggctctgtgggctccatgtagaagccactattactgggactgtgctcagagacccctctcccagctattcctactctctccccgactccgagagcatgcttaatcttgcttctgcttctcatttctgtagcctgatcagcgccgcaccagccgggaagagggtgattgctggggctcgtgccctgcatccctctcctcccagggcctgccccacagctcgggccctctgtgagatccgtctttggcctcctccagaatggagctggccctctcctggggatgtgtaatggtccccctgcttacccgcaaaagacaagtctttacagaatcaaatgcaattttaaatctgagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagtgtattttttcacttgtactaaaattgtatcaaatgtgacattatatgcactagcaataaaatgctaattgtttcatggtataaacgtcctactgtatgtgggaatttatttacctgaaataaaattcattagttgttagtgatggagcttaaaaaaaa

(19) United States
(12) Patent Application Publication
(10) Pub. No.: US 2016/0317569 A1
(43) Pub. Date: Nov. 3, 2016

(54) METHODS OF MANUFACTURING POLYSACCHARIDE COMPOSITIONS

(71) Applicant: MOMENTA PHARMACEUTICALS, INC., Cambridge, MA (US)

(72) Inventors: Congmei Zeng, Burlington, MA (US); Sean Smith, Littleton, MA (US); Simge Baloglu, Stoneham, MA (US); Birgit Schultes, Arlington, MA (US); James Anderson, Hudson, MA (US); Bharathi Govindarajan, Revere, MA (US)

(21) Appl. No.: 15/142,056

(22) Filed: Apr. 29, 2016

Related U.S. Application Data
(60) Provisional application No. 62/154,863, filed on Apr. 30, 2015.

(51) Int. Cl. A61K 31/727; G01N 33/50

(52) U.S. Cl. CPC A61K 31/727; G01N 33/5038; G01N 2400/40

(57) ABSTRACT

Methods of producing, processing, and manufacturing LMWHs.

### BACKGROUND

This disclosure relates to methods of manufacturing low molecular weight heparins (LMWHs).

### SUMMARY OF THE INVENTION

In one aspect, described herein are methods of manufacturing a LMWH (e.g., an M402) pharmaceutical composition, the method comprising: producing a batch of a LMWH preparation (e.g., a drug substance); determining for a sample of the batch whether the activity of the LMWH preparation is within a predetermined range by: exposing the sample to an assay comprising a population of cells that respond to a stimulant (e.g., a protein, e.g., a cytokine, e.g., SDF1α) in the absence of the LMWH within a predetermined first range; and determining, in the assay, whether the cellular response in the presence of the LMWH is within a predetermined second range (e.g., which is lower than said first range); and formulating the batch of LMWH (e.g., for medical use), to thereby manufacture a LMWH (e.g., an M402) pharmaceutical composition (e.g., a drug product).

In some embodiments, the cellular response is cellular migration. In some embodiments, the stimulant is a protein. In some embodiments, the stimulant is a cytokine. In some embodiments, the stimulant is SDF-1α.

In some embodiments, formulating comprises combining the batch of LMWH with a pharmaceutically acceptable carrier. In some embodiments, formulating comprises combining the batch of LMWH with one or more buffering agent (e.g., one or more buffering agent described herein (e.g., a phosphate buffer, a citrate buffer, a histidine buffer, a maleate buffer, a succinate buffer, an acetate buffer, or any combination thereof (e.g., a phosphate citrate buffer)).

In some embodiments, the buffering agent is a phosphate buffer (e.g., sodium phosphate), a citrate buffer (e.g., a sodium citrate buffer, e.g., 5-40 mM sodium citrate, e.g., 10-30 mM sodium citrate), a histidine buffer (e.g., 5-40 mM histidine, e.g., 10-30 mM histidine), a maleate buffer (e.g., 5-40 mM maleate buffer, e.g., 10-30 mM maleate buffer), a succinate buffer, an acetate buffer, or any combination thereof (e.g., a phosphate citrate buffer). In some embodiments, the buffering agent is a phosphate buffer (e.g., phosphate citrate; sodium phosphate). In some embodiments, the buffering agent is a phosphate citrate buffer.

In some embodiments, formulating further comprises adding one or more alcohol (e.g., benzyl alcohol, e.g., 15 mg/mL benzyl alcohol). In some embodiments, formulating further comprises adding one or more antioxidant (e.g., alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite).

In some embodiments, the pH of the pharmaceutical composition is between about 4.0-9.0 (e.g., between about 7.0-9.0). In some embodiments, the pH of the pharmaceutical composition is about 8.0 (e.g., about 7.5, 7.6, 7.7, 7.8, 7.9, 8.0, 8.1, 8.2, 8.3, 8.4, 8.5). In some embodiments, the buffering agent is a phosphate buffer (e.g., sodium phosphate) and the pH of the pharmaceutical composition is about 8.0 (e.g., about 7.5, 7.6, 7.7, 7.8, 7.9, 8.0, 8.1, 8.2, 8.3, 8.4, 8.5)

In some embodiments, the batch of the LMWH preparation is produced by a method comprising obtaining a preparation of unfractionated heparin (UFH); depolymerizing the UFH for a time and under conditions to obtain a first batch of LMWH having a weight average molecular weight of 3000-8000 Da; glycol splitting the first batch of LMWH to obtain a second batch of LMWH; and reducing and purifying the second batch of the LMWH, thereby producing the batch of the LMWH preparation.

In some embodiments, the LMWH preparation (e.g., the M402 preparation) comprises a polysaccharide of Formula (I)

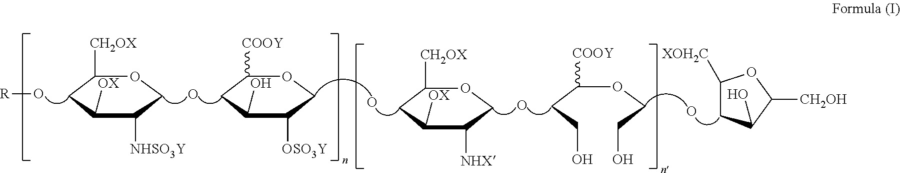

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

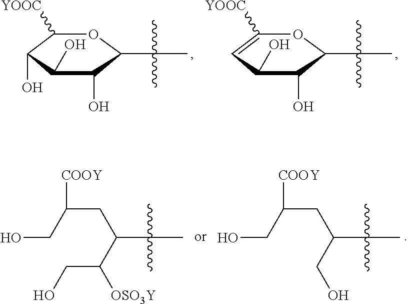

In some embodiments, the compound of Formula (I) is a compound of Formula (Ia)

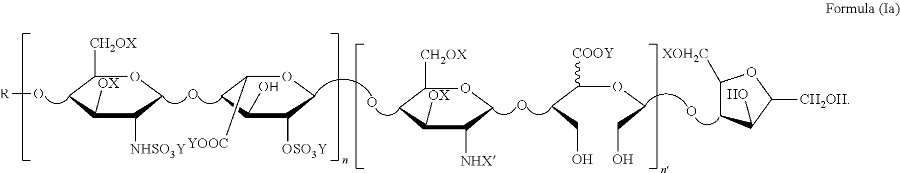

In some embodiments, Y for each occurrence is Na⁺.

In some embodiments, R is

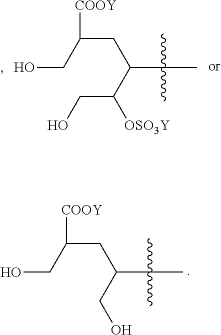

In some embodiments, the LMWH preparation (e.g., the M402 preparation) comprises or consists essentially of:

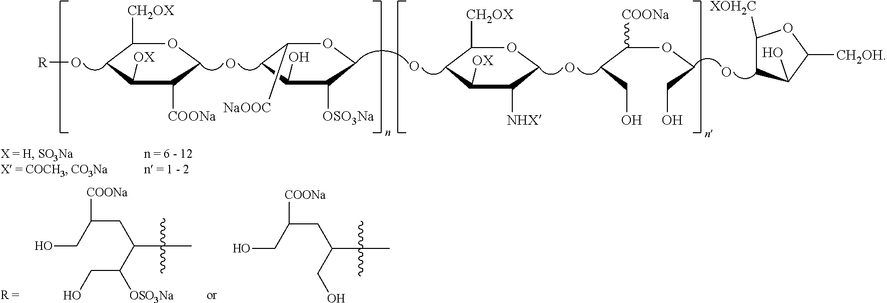

In some embodiments, the LMWH preparation is

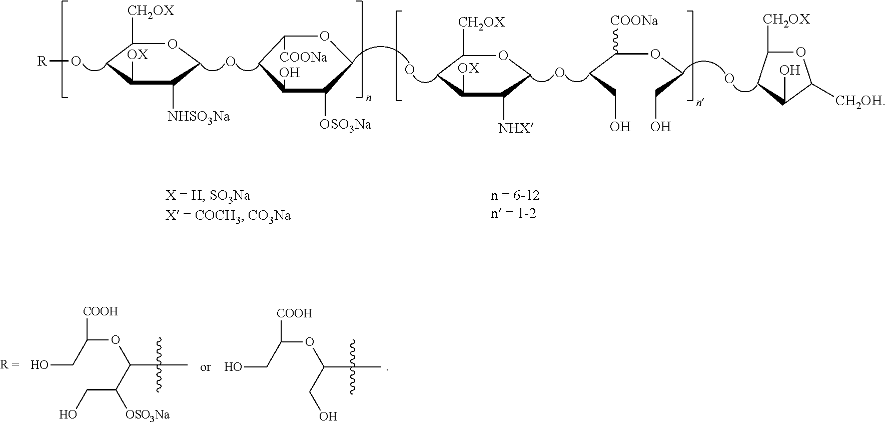

In some embodiments, the LMWH preparation (e.g., the M402 preparation) has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation (e.g., the M402 preparation) has the following characteristics: (a) a weight average chain molecular weight between 3,500 and 8,000 Da; (b) anti-Xa activity of less than 20 IU/mg and anti-IIa activity of less than 20 IU/mg; (c) greater than 5% and less than 25% glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In one aspect, described herein are methods of manufacturing a LMWH (e.g., an M402) pharmaceutical composition, the method comprising: producing a batch of a LMWH preparation (e.g., a drug substance); determining for a sample of the batch whether the activity of the LMWH preparation is within a predetermined range by: exposing the sample to an assay comprising a population of cells that migrate in response to a stimulant (e.g., a protein, e.g., a cytokine, e.g., SDF1α) in the absence of the LMWH within a predetermined first range; and determining, in the assay, whether the migration in the presence of the LMWH is within a predetermined second range (e.g., which is lower than said first range); and formulating the batch of LMWH (e.g., for medical use), to thereby manufacture a LMWH (e.g., an M402) pharmaceutical composition (e.g., a drug product).

In some embodiments, the stimulant is a protein. In some embodiments, the stimulant is a cytokine. In some embodiments, the stimulant is SDF-1α.

In some embodiments, formulating comprises combining the batch of LMWH with a pharmaceutically acceptable carrier. In some embodiments, formulating comprises combining the batch of LMWH with one or more buffering agent (e.g., one or more buffering agent described herein (e.g., a phosphate buffer, a citrate buffer, a histidine buffer, a maleate buffer, a succinate buffer, an acetate buffer, or any combination thereof (e.g., a phosphate citrate buffer)).

In some embodiments, the buffering agent is a phosphate buffer (e.g., sodium phosphate), a citrate buffer (e.g., a sodium citrate buffer, e.g., 5-40 mM sodium citrate, e.g., 10-30 mM sodium citrate), a histidine buffer (e.g., 5-40 mM histidine, e.g., 10-30 mM histidine), a maleate buffer (e.g., 5-40 mM maleate buffer, e.g., 10-30 mM maleate buffer), a succinate buffer, an acetate buffer, or any combination thereof (e.g., a phosphate citrate buffer). In some embodiments, the buffering agent is a phosphate buffer (e.g., phosphate citrate; sodium phosphate). In some embodiments, the buffering agent is a phosphate citrate buffer.

In some embodiments, formulating further comprises adding one or more alcohol (e.g., benzyl alcohol, e.g., 15 mg/mL benzyl alcohol). In some embodiments, formulating further comprises adding one or more antioxidant (e.g., alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite).

In some embodiments, the pH of the pharmaceutical composition is between about 4.0-9.0 (e.g., between about 7.0-9.0). In some embodiments, the pH of the pharmaceutical composition is about 8.0 (e.g., about 7.5, 7.6, 7.7, 7.8, 7.9, 8.0, 8.1, 8.2, 8.3, 8.4, 8.5). In some embodiments, the buffering agent is a phosphate buffer (e.g., sodium phosphate) and the pH of the pharmaceutical composition is about 8.0 (e.g., about 7.5, 7.6, 7.7, 7.8, 7.9, 8.0, 8.1, 8.2, 8.3, 8.4, 8.5).

In some embodiments, the batch of the LMWH preparation is produced by a method comprising obtaining a preparation of unfractionated heparin (UFH); depolymerizing the UFH for a time and under conditions to obtain a first batch of LMWH having a weight average molecular weight of 3000-8000 Da; glycol splitting the first batch of LMWH to obtain a second batch of LMWH; and reducing and purifying the second batch of the LMWH, thereby producing the batch of the LMWH preparation.

In some embodiments, the LMWH preparation (e.g., the M402 preparation) comprises a polysaccharide of Formula (I)

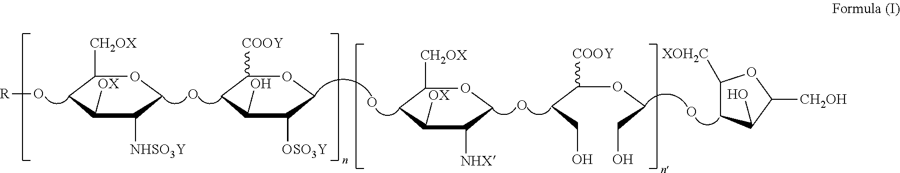

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

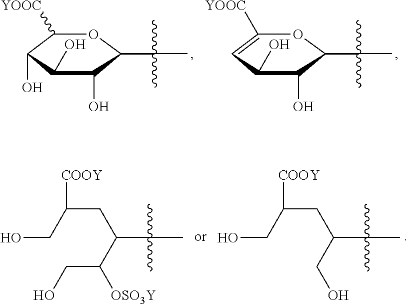

In some embodiments, the compound of Formula (I) is a compound of Formula (Ia)

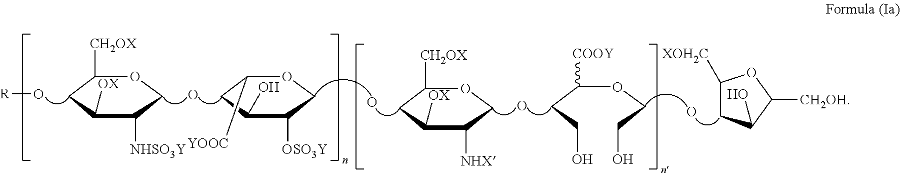

In some embodiments, Y for each occurrence is Na⁺.

In some embodiments, R is

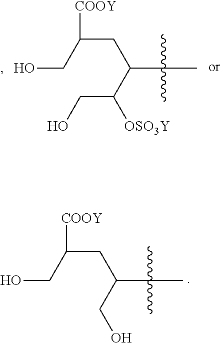

In some embodiments, the LMWH preparation (e.g., the M402 preparation) comprises or consists essentially of:

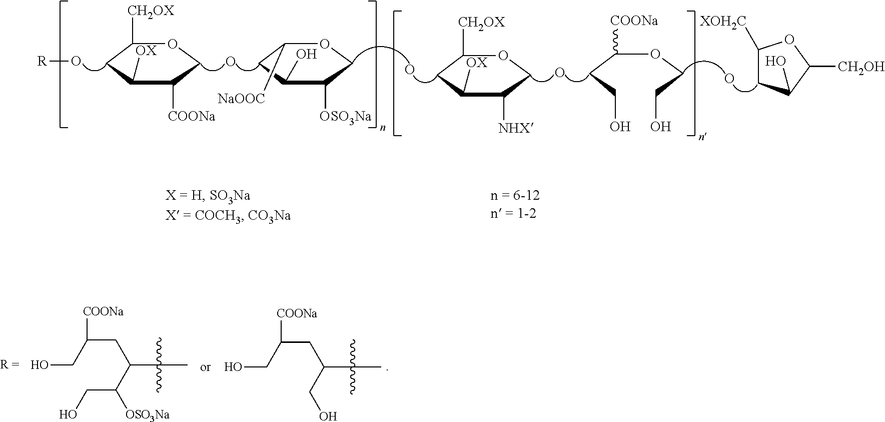

In some embodiments, the LMWH preparation is

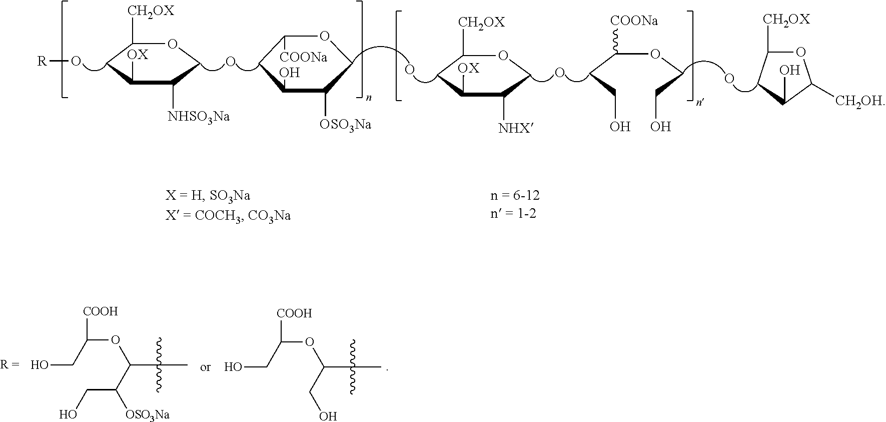

In some embodiments, the LMWH preparation (e.g., the M402 preparation) has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation (e.g., the M402 preparation) has the following characteristics: (a) a weight average chain molecular weight between 3,500 and 8,000 Da; (b) anti-Xa activity of less than 20 IU/mg and anti-IIa activity of less than 20 IU/mg; (c) greater than 5% and less than 25% glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In one aspect, described herein are methods of manufacturing a LMWH (e.g., an M402) pharmaceutical composition, the method comprising: obtaining a preparation of unfractionated heparin (UFH); depolymerizing the UFH for a time and under conditions to obtain a first batch of LMWH having a weight average molecular weight of 3000-8000 Da; glycol splitting the first batch of LMWH to obtain a second batch of LMWH; reducing and purifying the second batch of the LMWH; determining for a sample of the batch whether the activity of the LMWH preparation is within a predetermined range by: exposing the sample to an assay, wherein the assay comprises a population of cells that respond to a stimulant (e.g., a protein, e.g., a cytokine, e.g., SDF1α) in the absence of the LMWH within a predetermined first range; and determining, in the assay, whether the cellular response in the presence of the LMWH is within a predetermined second range; and formulating the batch of LMWH (e.g., for medical use), to thereby manufacture a LMWH (e.g., M402) pharmaceutical composition.

In some embodiments, the cellular response is cellular migration. In some embodiments, the stimulant is a protein. In some embodiments, the stimulant is a cytokine. In some embodiments, the stimulant is SDF-1α.

In some embodiments, formulating comprises combining the batch of LMWH with a pharmaceutically acceptable carrier. In some embodiments, formulating comprises combining the batch of LMWH with one or more buffering agent (e.g., one or more buffering agent described herein (e.g., a phosphate buffer, a citrate buffer, a histidine buffer, a maleate buffer, a succinate buffer, an acetate buffer, or any combination thereof (e.g., a phosphate citrate buffer)).

In some embodiments, the buffering agent is a phosphate buffer (e.g., sodium phosphate), a citrate buffer (e.g., a sodium citrate buffer, e.g., 5-40 mM sodium citrate, e.g., 10-30 mM sodium citrate), a histidine buffer (e.g., 5-40 mM histidine, e.g., 10-30 mM histidine), a maleate buffer (e.g., 5-40 mM maleate buffer, e.g., 10-30 mM maleate buffer), a succinate buffer, an acetate buffer, or any combination thereof (e.g., a phosphate citrate buffer). In some embodiments, the buffering agent is a phosphate buffer (e.g., phosphate citrate; sodium phosphate). In some embodiments, the buffering agent is a phosphate citrate buffer.

In some embodiments, formulating further comprises adding one or more alcohol (e.g., benzyl alcohol, e.g., 15 mg/mL benzyl alcohol). In some embodiments, formulating further comprises adding one or more antioxidant (e.g., alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite).

In some embodiments, the pH of the pharmaceutical composition is between about 4.0-9.0 (e.g., between about 7.0-9.0). In some embodiments, the pH of the pharmaceutical composition is about 8.0 (e.g., about 7.5, 7.6, 7.7, 7.8, 7.9, 8.0, 8.1, 8.2, 8.3, 8.4, 8.5). In some embodiments, the buffering agent is a phosphate buffer (e.g., sodium phosphate) and the pH of the pharmaceutical composition is about 8.0 (e.g., about 7.5, 7.6, 7.7, 7.8, 7.9, 8.0, 8.1, 8.2, 8.3, 8.4, 8.5).

In some embodiments, the batch of the LMWH preparation is produced by a method comprising obtaining a preparation of unfractionated heparin (UFH); depolymerizing the UFH for a time and under conditions to obtain a first batch of LMWH having a weight average molecular weight of 3000-8000 Da; glycol splitting the first batch of LMWH to obtain a second batch of LMWH; and reducing and purifying the second batch of the LMWH, thereby producing the batch of the LMWH preparation.

In some embodiments, the LMWH preparation (e.g., the M402 preparation) comprises a polysaccharide of Formula (I)

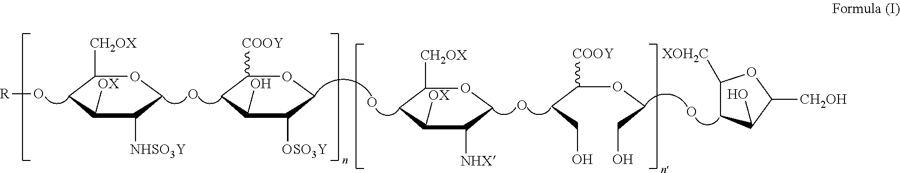

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

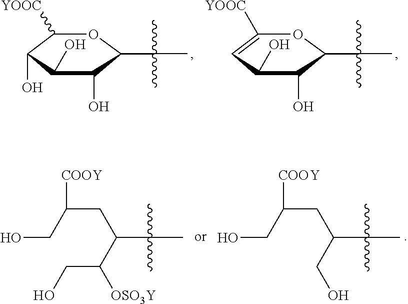

In some embodiments, the compound of Formula (I) is a compound of Formula (Ia)

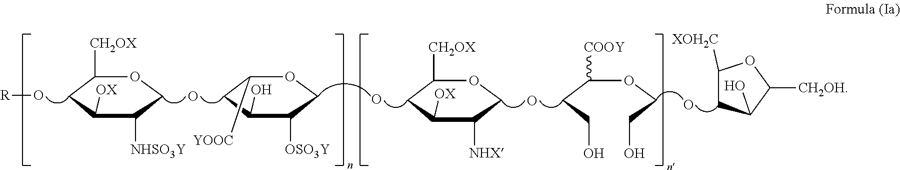

In some embodiments, Y for each occurrence is Na⁺.

In some embodiments, R is

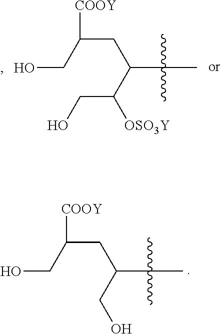

In some embodiments, the LMWH preparation (e.g., the M402 preparation) comprises or consists essentially of:

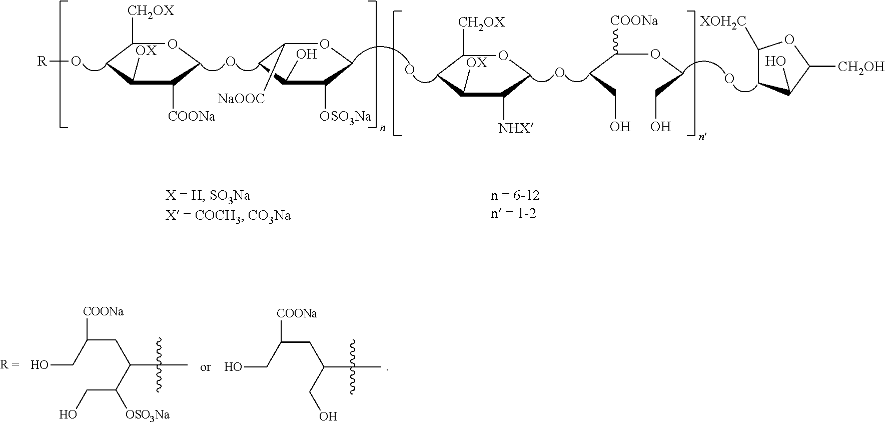

In some embodiments, the LMWH preparation is

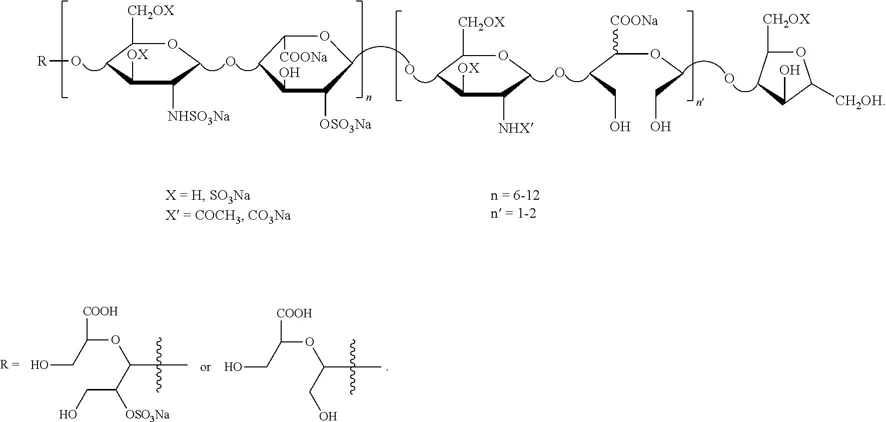

In some embodiments, the LMWH preparation (e.g., the M402 preparation) has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation (e.g., the M402 preparation) has the following characteristics: (a) a weight average chain molecular weight between 3,500 and 8,000 Da; (b) anti-Xa activity of less than 20 IU/mg and anti-IIa activity of less than 20 IU/mg; (c) greater than 5% and less than 25% glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In one aspect, described herein are methods of processing a LMWH preparation, the method comprising: determining for a sample of the batch whether the potency of the LMWH preparation is within a predetermined range by: exposing the sample to an assay, wherein the assay comprises a population of cells that respond to a stimulant (e.g., a protein, e.g., a cytokine, e.g., SDF1α) in the absence of the LMWH within a predetermined first range; and determining, in the assay, whether the cellular response in the presence of the LMWH is within a predetermined second range; and if cells response is within the predetermined second range, processing the preparation, wherein processing includes one or more of: releasing, classifying, selecting, accepting, processing into a drug product, shipping, formulating, labeling, packaging, releasing into commerce and selling the preparation.

In some embodiments, the cellular response is cellular migration. In some embodiments, the stimulant is a protein. In some embodiments, the stimulant is a cytokine. In some embodiments, the stimulant is SDF-1α.

In some embodiments, the processing includes one or more of processing into a drug product, shipping, formulating, labeling, packaging.

In some embodiments, the LMWH preparation (e.g., the M402 preparation) comprises a polysaccharide of Formula (I)

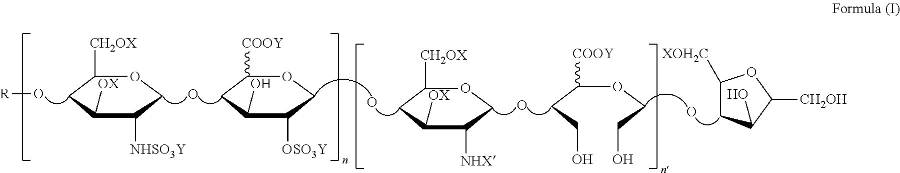

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

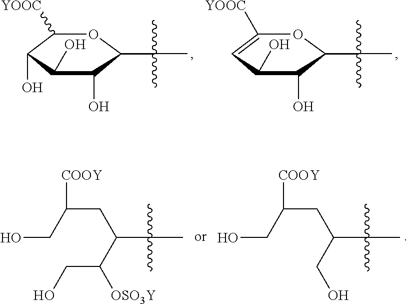

In some embodiments, the compound of Formula (I) is a compound of Formula (Ia)

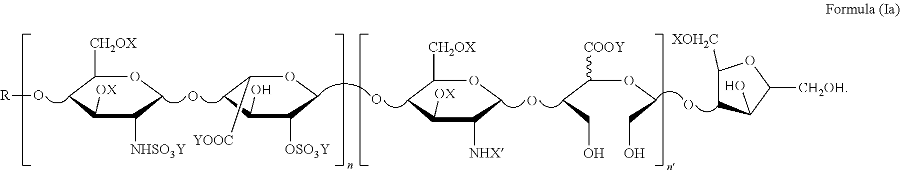

In some embodiments, Y for each occurrence is Na⁺.

In some embodiments, R is

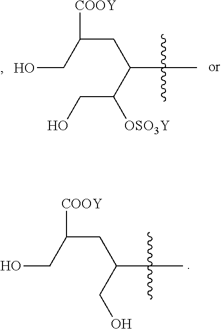

In some embodiments, the LMWH preparation (e.g., the M402 preparation) comprises or consists essentially of:

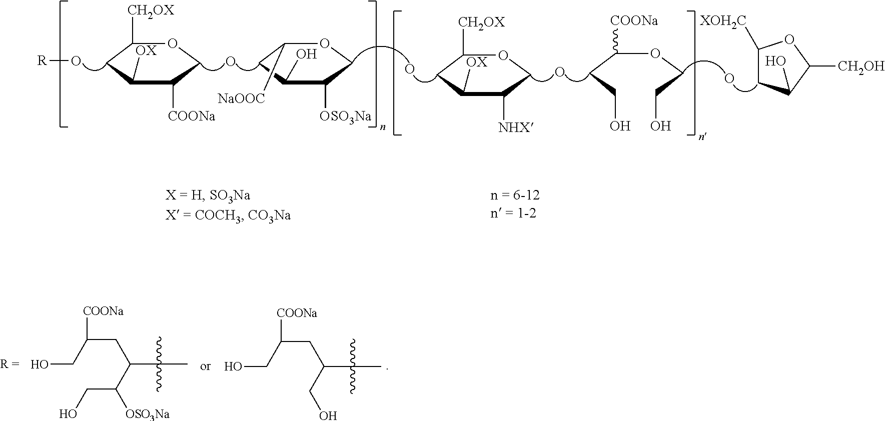

In some embodiments, the LMWH preparation is

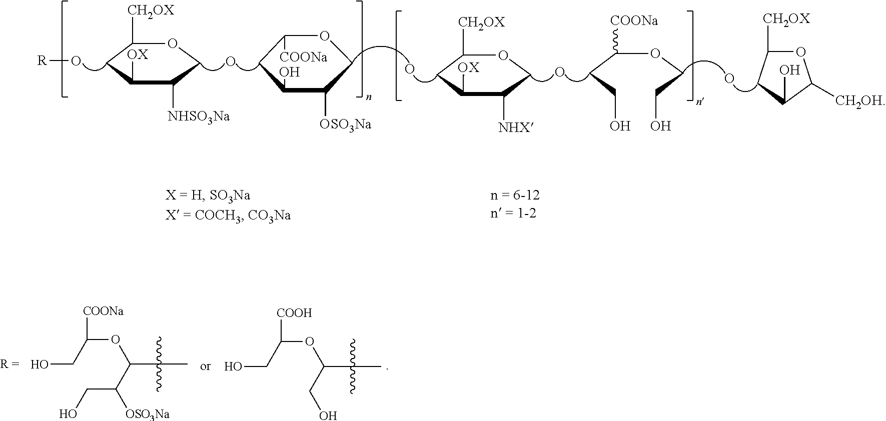

In some embodiments, the LMWH preparation (e.g., the M402 preparation) has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation (e.g., the M402 preparation) has the following characteristics: (a) a weight average chain molecular weight between 3,500 and 8,000 Da; (b) anti-Xa activity of less than 20 IU/mg and anti-IIa activity of less than 20 IU/mg; (c) greater than 5% and less than 25% glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In one aspect, described herein are methods of manufacturing an M402 pharmaceutical composition, the composition comprising a polysaccharide of Formula (I)

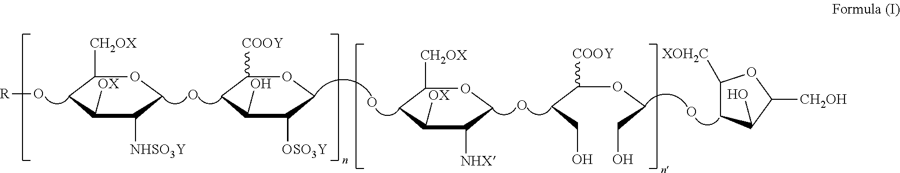

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

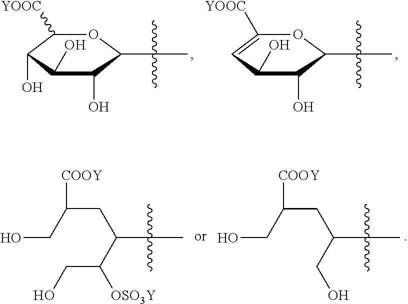

the method comprising:

producing or obtaining a batch of a LMWH preparation;

determining for a sample of the batch whether the activity of the LMWH preparation is within a predetermined range by: exposing the sample to an assay, wherein the assay comprises a population of cells that respond to a stimulant (e.g., a protein, e.g., a cytokine, e.g., SDF1α) in the absence of the LMWH within a first range; and determining, in the assay, whether the cellular response in the presence of the LMWH is within a predetermined second range; and formulating the batch of LMWH (e.g., for medical use), to thereby manufacture a LMWH (e.g., M402) pharmaceutical composition.

In some embodiments, the cellular response is cellular migration. In some embodiments, the stimulant is a protein. In some embodiments, the stimulant is a cytokine. In some embodiments, the stimulant is SDF-1α.

In some embodiments, formulating comprises combining the batch of LMWH with a pharmaceutically acceptable carrier. In some embodiments, formulating comprises combining the batch of LMWH with one or more buffering agent (e.g., one or more buffering agent described herein (e.g., a phosphate buffer, a citrate buffer, a histidine buffer, a maleate buffer, a succinate buffer, an acetate buffer, or any combination thereof (e.g., a phosphate citrate buffer)).

In some embodiments, the buffering agent is a phosphate buffer (e.g., sodium phosphate), a citrate buffer (e.g., a sodium citrate buffer, e.g., 5-40 mM sodium citrate, e.g., 10-30 mM sodium citrate), a histidine buffer (e.g., 5-40 mM histidine, e.g., 10-30 mM histidine), a maleate buffer (e.g., 5-40 mM maleate buffer, e.g., 10-30 mM maleate buffer), a succinate buffer, an acetate buffer, or any combination thereof (e.g., a phosphate citrate buffer). In some embodiments, the buffering agent is a phosphate buffer (e.g., phosphate citrate; sodium phosphate). In some embodiments, the buffering agent is a phosphate citrate buffer.

In some embodiments, formulating further comprises adding one or more alcohol (e.g., benzyl alcohol, e.g., 15 mg/mL benzyl alcohol). In some embodiments, formulating further comprises adding one or more antioxidant (e.g., alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite).

In some embodiments, the pH of the pharmaceutical composition is between about 4.0-9.0 (e.g., between about 7.0-9.0). In some embodiments, the pH of the pharmaceutical composition is about 8.0 (e.g., about 7.5, 7.6, 7.7, 7.8, 7.9, 8.0, 8.1, 8.2, 8.3, 8.4, 8.5). In some embodiments, the buffering agent is a phosphate buffer (e.g., sodium phosphate) and the pH of the pharmaceutical composition is about 8.0 (e.g., about 7.5, 7.6, 7.7, 7.8, 7.9, 8.0, 8.1, 8.2, 8.3, 8.4, 8.5).

In one aspect, described herein are methods of manufacturing an M402 pharmaceutical composition, the composition comprising a polysaccharide of Formula (I)

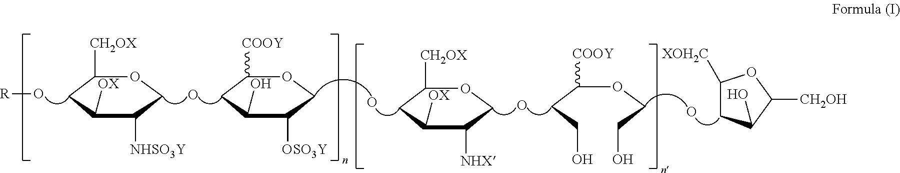

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

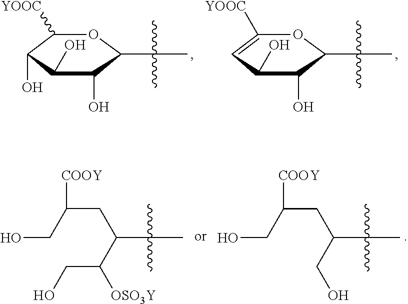

the method comprising:

obtaining a preparation of unfractionated heparin (UFH);

depolymerizing the UFH for a time and under conditions to obtain a first batch of LMWH having a weight average molecular weight of 3000-8000 Da;

glycol splitting the first batch of LMWH to obtain a second batch of LMWH;

reducing and purifying the second batch of the LMWH;

determining for a sample of the batch whether the potency of the LMWH preparation is within a predetermined range by: exposing the sample to an assay, wherein the assay comprises a population of cells that respond to a stimulant (e.g., a protein, e.g., a cytokine, e.g., SDF1α) in the absence of the LMWH within a predetermined first range; and determining, in the assay, whether the cellular response response in the presence of the LMWH is within a predetermined second range; and formulating the batch of LMWH (e.g., for medical use), to thereby manufacture a LMWH (e.g., M402) pharmaceutical composition. In some embodiments, the cellular response is migration.

In some embodiments, the cellular response is cellular migration. In some embodiments, the stimulant is a protein. In some embodiments, the stimulant is a cytokine. In some embodiments, the stimulant is SDF-1α.

In some embodiments, formulating comprises combining the batch of LMWH with a pharmaceutically acceptable carrier. In some embodiments, formulating comprises combining the batch of LMWH with one or more buffering agent (e.g., one or more buffering agent described herein (e.g., a phosphate buffer, a citrate buffer, a histidine buffer, a maleate buffer, a succinate buffer, an acetate buffer, or any combination thereof (e.g., a phosphate citrate buffer)).

In some embodiments, the buffering agent is a phosphate buffer (e.g., sodium phosphate), a citrate buffer (e.g., a sodium citrate buffer, e.g., 5-40 mM sodium citrate, e.g., 10-30 mM sodium citrate), a histidine buffer (e.g., 5-40 mM histidine, e.g., 10-30 mM histidine), a maleate buffer (e.g., 5-40 mM maleate buffer, e.g., 10-30 mM maleate buffer), a succinate buffer, an acetate buffer, or any combination thereof (e.g., a phosphate citrate buffer). In some embodiments, the buffering agent is a phosphate buffer (e.g., phosphate citrate; sodium phosphate). In some embodiments, the buffering agent is a phosphate citrate buffer.

In some embodiments, formulating further comprises adding one or more alcohol (e.g., benzyl alcohol, e.g., 15 mg/mL benzyl alcohol). In some embodiments, formulating further comprises adding one or more antioxidant (e.g., alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite).

In some embodiments, the pH of the pharmaceutical composition is between about 4.0-9.0 (e.g., between about 7.0-9.0). In some embodiments, the pH of the pharmaceutical composition is about 8.0 (e.g., about 7.5, 7.6, 7.7, 7.8, 7.9, 8.0, 8.1, 8.2, 8.3, 8.4, 8.5). In some embodiments, the buffering agent is a phosphate buffer (e.g., sodium phosphate) and the pH of the pharmaceutical composition is about 8.0 (e.g., about 7.5, 7.6, 7.7, 7.8, 7.9, 8.0, 8.1, 8.2, 8.3, 8.4, 8.5).

In one aspect, described herein are methods of manufacturing a LMWH (e.g., M402) pharmaceutical composition, the method comprising: determining for a sample of the batch whether the activity of the LMWH preparation is within a predetermined range by: exposing the sample to an assay, wherein the assay comprises a population of cells that respond to a stimulant (e.g., a protein, e.g., a cytokine, e.g., SDF1α) in the absence of the LMWH within a predetermined first range, in response; and determining, in the assay, whether the cellular response in the presence of the LMWH is within a predetermined second range; and formulating the batch of LMWH (e.g., for medical use), to thereby manufacture a LMWH (e.g., M402) pharmaceutical composition.

In some embodiments, the cellular response is cellular migration. In some embodiments, the stimulant is a protein. In some embodiments, the stimulant is a cytokine. In some embodiments, the stimulant is SDF-1α.

In some embodiments, formulating comprises combining the batch of LMWH with a pharmaceutically acceptable carrier. In some embodiments, formulating comprises combining the batch of LMWH with one or more buffering agent (e.g., one or more buffering agent described herein (e.g., a phosphate buffer, a citrate buffer, a histidine buffer, a maleate buffer, a succinate buffer, an acetate buffer, or any combination thereof (e.g., a phosphate citrate buffer)).

In some embodiments, the buffering agent is a phosphate buffer (e.g., sodium phosphate), a citrate buffer (e.g., a sodium citrate buffer, e.g., 5-40 mM sodium citrate, e.g., 10-30 mM sodium citrate), a histidine buffer (e.g., 5-40 mM histidine, e.g., 10-30 mM histidine), a maleate buffer (e.g., 5-40 mM maleate buffer, e.g., 10-30 mM maleate buffer), a succinate buffer, an acetate buffer, or any combination thereof (e.g., a phosphate citrate buffer). In some embodiments, the buffering agent is a phosphate buffer (e.g., phosphate citrate; sodium phosphate). In some embodiments, the buffering agent is a phosphate citrate buffer.

In some embodiments, formulating further comprises adding one or more alcohol (e.g., benzyl alcohol, e.g., 15 mg/mL benzyl alcohol). In some embodiments, formulating further comprises adding one or more antioxidant (e.g., alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite).

In some embodiments, the pH of the pharmaceutical composition is between about 4.0-9.0 (e.g., between about 7.0-9.0). In some embodiments, the pH of the pharmaceutical composition is about 8.0 (e.g., about 7.5, 7.6, 7.7, 7.8, 7.9, 8.0, 8.1, 8.2, 8.3, 8.4, 8.5). In some embodiments, the buffering agent is a phosphate buffer (e.g., sodium phosphate) and the pH of the pharmaceutical composition is about 8.0 (e.g., about 7.5, 7.6, 7.7, 7.8, 7.9, 8.0, 8.1, 8.2, 8.3, 8.4, 8.5)

In some embodiments, the LMWH preparation (e.g., the M402 preparation) comprises a polysaccharide of Formula (I)

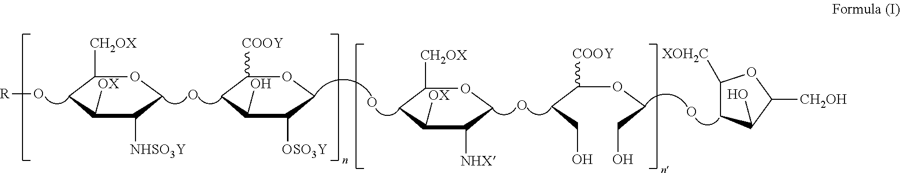

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

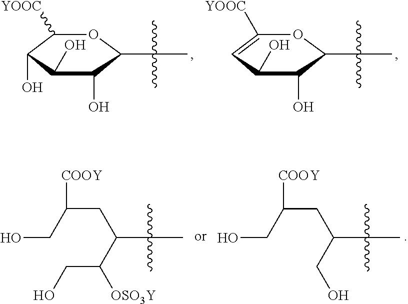

In some embodiments, the compound of Formula (I) is a compound of Formula (Ia)

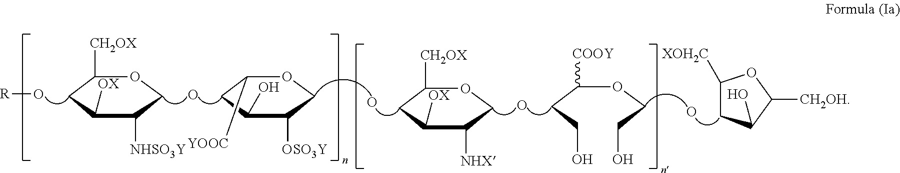

In some embodiments, Y for each occurrence is Na⁺.

In some embodiments, R is

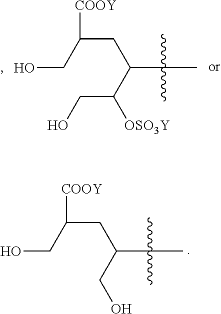

In some embodiments, the LMWH preparation (e.g., the M402 preparation) comprises or consists essentially of:

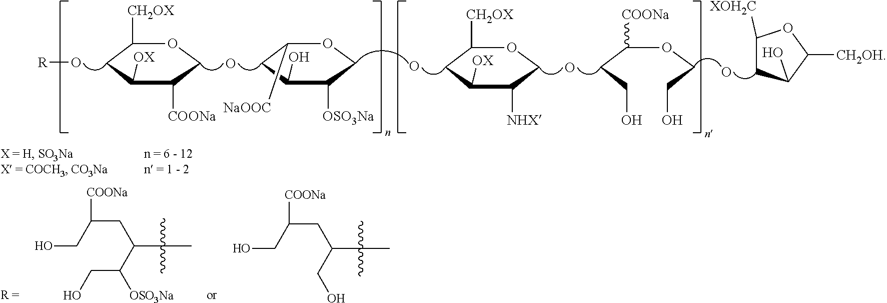

In some embodiments, the LMWH preparation is

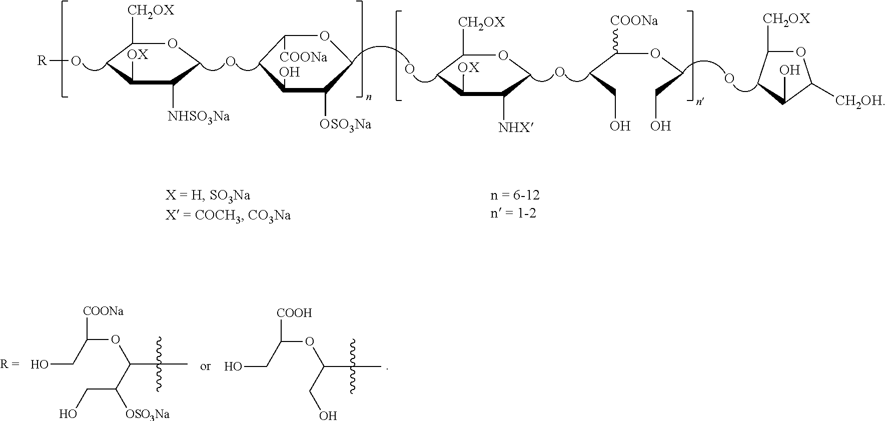

In some embodiments, the LMWH preparation (e.g., the M402 preparation) has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation (e.g., the M402 preparation) has the following characteristics: (a) a weight average chain molecular weight between 3,500 and 8,000 Da; (b) anti-Xa activity of less than 20 IU/mg and anti-IIa activity of less than 20 IU/mg; (c) greater than 5% and less than 25% glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In one aspect, described herein are methods of manufacturing a unit dosage form of a LMWH (e.g., an M402) composition (e.g., a pharmaceutical composition) for medical use, the method comprising: producing a batch of a LMWH pharmaceutical composition; exposing the sample to an assay, wherein the assay comprises a population of cells that respond to a stimulant (e.g., a protein, e.g., a cytokine, e.g., SDF1α) in the absence of the LMWH within a predetermined first range; and determining, in the assay, whether the cellular response in the presence of the LMWH is within a predetermined second range; and preparing the LMWH composition as a unit dosage form, thereby manufacturing a unit dosage form of a LMWH (e.g., M402) composition.

In some embodiments, the cellular response is cellular migration. In some embodiments, the stimulant is a protein. In some embodiments, the stimulant is a cytokine. In some embodiments, the stimulant is SDF-1α.

In some embodiments, preparing the LMWH composition as a unit dosage form comprises one or more of packaging, labeling, or shipping the selected batch of LMWH (e.g., M402). In some embodiments, preparing the LMWH composition as a unit dosage form comprises packaging the selected batch of LMWH (e.g., M402) into a container or vial e.g., comprising an anaerobic overlay, e.g., a nitrogen overlay. In some embodiments, preparing the LMWH composition as a unit dosage form comprises labeling the selected batch of LMWH at a dose between 1-40 mg/kg (e.g., between 1-20 mg/kg, between 2-20 mg/kg, between 2-10 mg/kg, between 2-8 mg/kg)).

In some embodiments, the LMWH preparation (e.g., the M402 preparation) comprises a polysaccharide of Formula (I)

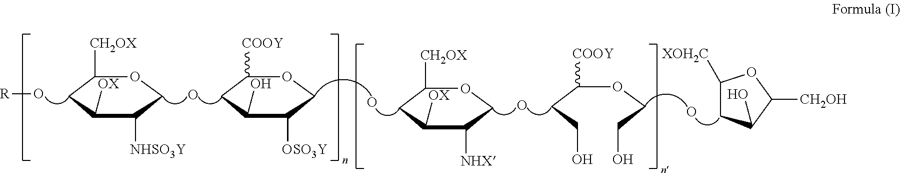

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

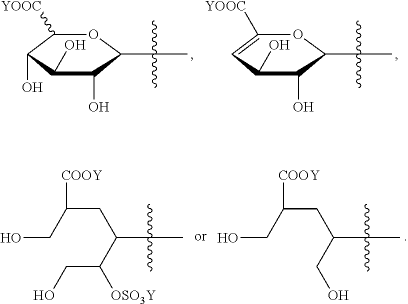

In some embodiments, the compound of Formula (I) is a compound of Formula (Ia)

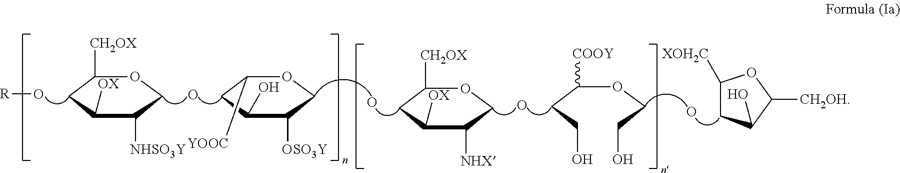

In some embodiments, Y for each occurrence is Na⁺.

In some embodiments, R is

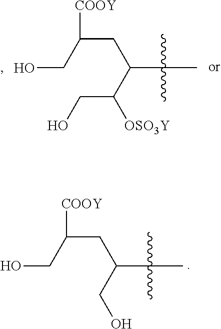

In some embodiments, the LMWH preparation (e.g., the M402 preparation) comprises or consists essentially of:

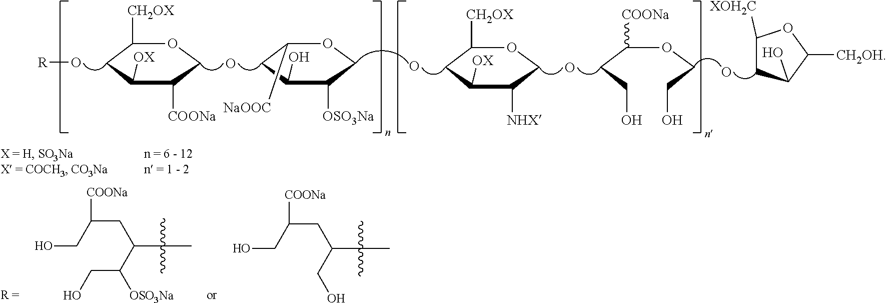

In some embodiments, the LMWH preparation is an M402 preparation.

In some embodiments, the LMWH preparation is

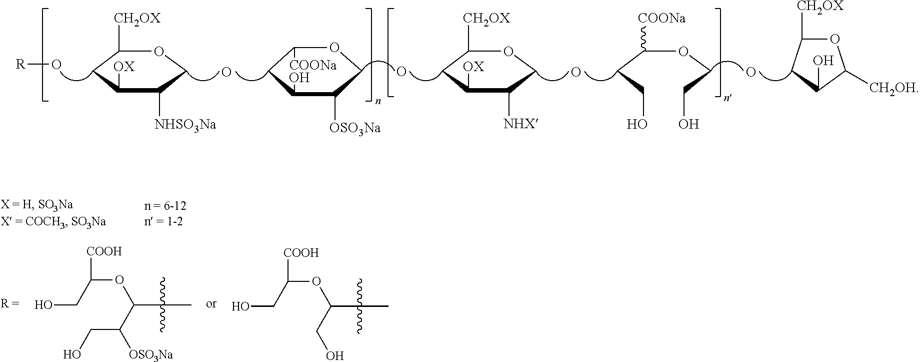

In some embodiments, the LMWH preparation (e.g., the M402 preparation) has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation (e.g., the M402 preparation) has the following characteristics: (a) a weight average chain molecular weight between 3,500 and 8,000 Da; (b) anti-Xa activity of less than 20 IU/mg and anti-IIa activity of less than 20 IU/mg; (c) greater than 5% and less than 25% glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In one aspect, described herein are methods of manufacturing a unit dosage form of an M402 pharmaceutical composition, the composition comprising a polysaccharide of Formula (I)

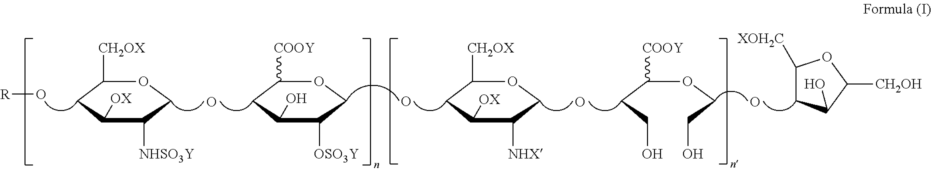

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

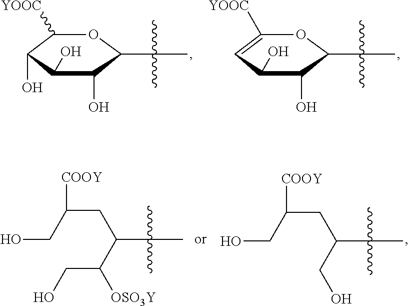

the method comprising:

producing or obtaining a batch of an M402 pharmaceutical composition; exposing the sample to an assay, wherein the potency assay comprises a population of cells that respond to a stimulant (e.g., a protein, e.g., a cytokine, e.g., SDF1α) in the absence of the LMWH within a predetermined first range; and determining, in the assay, whether the cellular response in the presence of the LMWH is within a predetermined second range; and preparing the LMWH composition as a unit dosage form, thereby manufacturing a unit dosage form of a LMWH (e.g., M402) composition.

In some embodiments, the cellular response is cellular migration. In some embodiments, the stimulant is a protein. In some embodiments, the stimulant is a cytokine. In some embodiments, the stimulant is SDF-1α.

In some embodiments, preparing the LMWH composition as a unit dosage form comprises one or more of packaging, labeling, or shipping the selected batch of LMWH (e.g., M402). In some embodiments, preparing the LMWH composition as a unit dosage form comprises packaging the selected batch of LMWH (e.g., M402) into a container or vial e.g., comprising an anaerobic overlay, e.g., a nitrogen overlay. In some embodiments, preparing the LMWH composition as a unit dosage form comprises labeling the selected batch of LMWH at a dose between 1-40 mg/kg (e.g., between 1-20 mg/kg, between 2-20 mg/kg, between 2-10 mg/kg, between 2-8 mg/kg)).

In one aspect, described herein are methods of manufacturing a unit dosage form of a LMWH (e.g., M402) (e.g., a pharmaceutical composition) for medical use, the method comprising: producing a batch of a necuparanib pharmaceutical composition; exposing the sample to an assay, wherein the assay comprises a population of cells that respond to a stimulant (e.g., a protein, e.g., a cytokine, e.g., SDF1α) in the absence of the LMWH within a predetermined first range; and determining, in the assay, whether the cellular response in the presence of the LMWH is within a predetermined second range; and preparing the LMWH composition as a unit dosage form, thereby manufacturing a unit dosage form of a LMWH (e.g., M402) composition.

In some embodiments, the cellular response is cellular migration. In some embodiments, the stimulant is a protein. In some embodiments, the stimulant is a cytokine. In some embodiments, the stimulant is SDF-1α.

In some embodiments, preparing the LMWH composition as a unit dosage form comprises one or more of packaging, labeling, or shipping the selected batch of LMWH (e.g., M402). In some embodiments, preparing the LMWH composition as a unit dosage form comprises packaging the selected batch of LMWH (e.g., M402) into a container or vial e.g., comprising an anaerobic overlay, e.g., a nitrogen overlay. In some embodiments, preparing the LMWH composition as a unit dosage form comprises labeling the selected batch of LMWH at a dose between 1-40 mg/kg (e.g., between 1-20 mg/kg, between 2-20 mg/kg, between 2-10 mg/kg, between 2-8 mg/kg)).

In one aspect, described herein are methods of determining the activity (e.g., potency) of a LMWH (e.g., M402) preparation or composition, the method comprising: providing a plurality of cells (e.g., Jurkat cells) on a substrate, wherein the cells migrate in response to a stimulant (e.g., a protein, e.g., a cytokine, e.g., SDF1α) in the absence of the LMWH within a first range; providing the stimulant (e.g., a protein, e.g., a cytokine, e.g., a SDF-1α); adding the LMWH (e.g., M402) preparation; incubating the cells for a period of time (e.g., at least 1 hour, e.g., at least 2, 4, 6, 8, 10, 12, 24 hours); detecting cell migration of at least one cell across the substrate; and determining, whether cell migration in the presence of the LMWH is within a predetermined second range.

In some embodiments, the cells are mammalian cells (e.g., Jurkat cells). In some embodiments, the stimulant is a protein. In some embodiments, the stimulant is a cytokine (e.g., SDF-1a). In some embodiments, the cells are incubated for at least 1 hour (e.g., at least 2, 4, 6, 8, 10, 12, 24 hours). In some embodiments, the cellular movement is detected using a metabolic dye. In some embodiments, the LMWH (e.g., M402) preparation is added at a concentration between 1 to 50 ng/ml (e.g., 4 ng/ml).

In some embodiments, the LMWH preparation (e.g., the M402 preparation) comprises a polysaccharide of Formula (I)

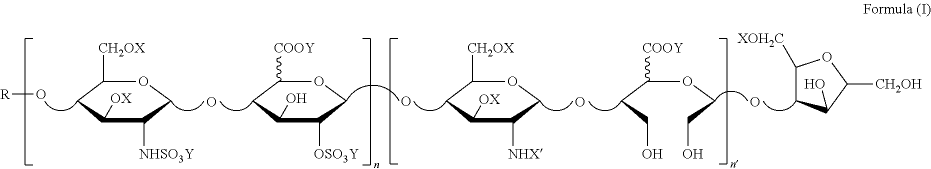

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

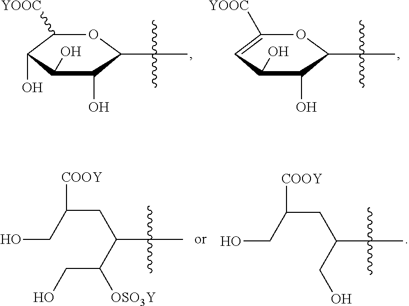

In some embodiments, the compound of Formula (I) is a compound of Formula (Ia)

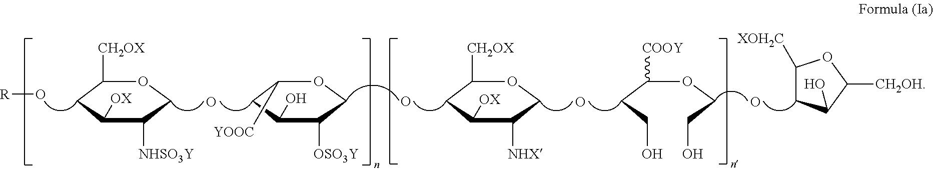

In some embodiments, Y for each occurrence is Na⁺.

In some embodiments, R is

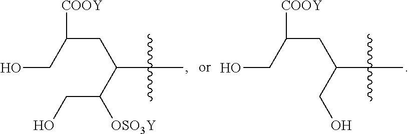

In some embodiments, the LMWH preparation (e.g., the M402 preparation) comprises or consists essentially of:

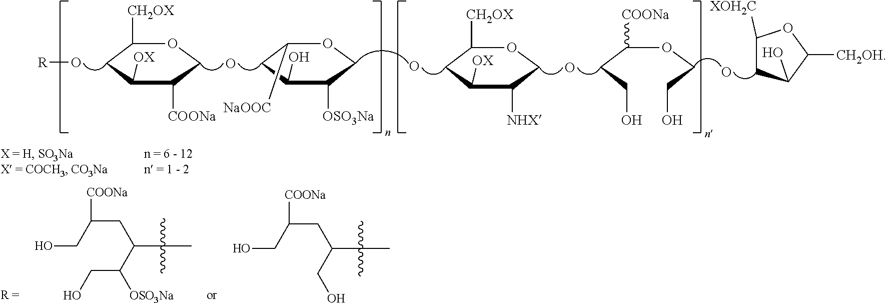

In some embodiments, the LMWH preparation is

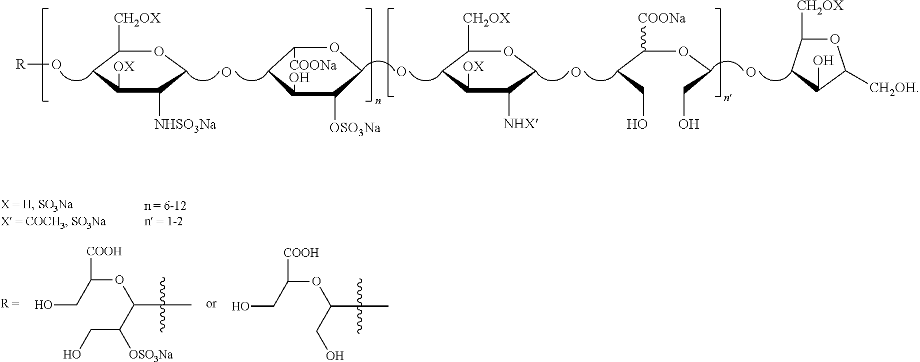

In some embodiments, the LMWH preparation (e.g., the M402 preparation) has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation (e.g., the M402 preparation) has the following characteristics: (a) a weight average chain molecular weight between 3,500 and 8,000 Da; (b) anti-Xa activity of less than 20 IU/mg and anti-IIa activity of less than 20 IU/mg; (c) greater than 5% and less than 25% glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In one aspect, described herein are methods of determining the potency of a LMWH (e.g., M402) preparation, the preparation comprising a polysaccharide of Formula (I)

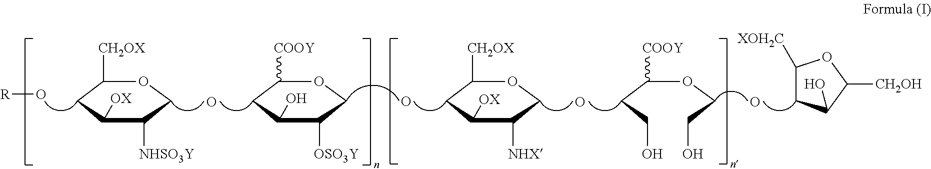

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

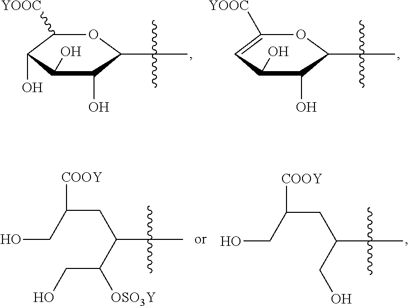

the method comprising:

providing a plurality of cells (e.g., Jurkat cells) on a substrate, wherein the cells migrate in response to a stimulant (e.g., a protein, e.g., a cytokine, e.g., SDF1α) in the absence of the LMWH within a first range; providing the stimulant (e.g., a protein, e.g., a cytokine, e.g., a SDF-1a); adding the LMWH (e.g., M402) preparation; incubating the cells for a period of time (e.g., at least 1 hour, e.g., at least 2, 4, 6, 8, 10, 12, 24 hours); detecting cell migration of at least one cell across the substrate; and determining, whether cell migration in the presence of the LMWH is within a predetermined second range.

In some embodiments, the cells are mammalian cells (e.g., Jurkat cells). In some embodiments, the stimulant is a protein. In some embodiments, the stimulant is a cytokine (e.g., SDF-1a). In some embodiments, the cells are incubated for at least 1 hour (e.g., at least 2, 4, 6, 8, 10, 12, 24 hours). In some embodiments, the cellular movement is detected using a metabolic dye. In some embodiments, the LMWH (e.g., M402) preparation is added at a concentration between 1 to 10000 μg/ml.

### DETAILED DESCRIPTION

The present disclosure provides methods for producing, e.g., manufacturing (e.g., manufacturing or controlling, monitoring, and/or assessing the manufacture of) LMWH, such as M402. For example, methods disclosed herein can be used to manufacture LMWH that qualifies as M402. The disclosure is based, in part, on the discovery that stimulant induced cell migration can be modulated by certain LMWHs and that the level of modulation can serve as a proxy for identification of the LMWH.

Methods described herein include manufacturing a batch of LMWH, during the manufacturing process obtaining a sample of the batch of the LMWH, assessing the sample using the methods disclosed herein, and continuing the manufacture if (e.g., only if) the sample qualifies as M402 or an M402 precursor. In some instances, methods include determining (e.g., detecting) whether a sample (e.g., a sample of a manufactured batch) qualifies as M402. For example, methods include exposing a sample of a batch of a LMWH preparation or composition to an assay, wherein the assay comprises a population of cells that migrate within a predetermined first range, in response to a stimulant (e.g., SDF1α) in the absence of the LMWH; and determining (e.g., detecting), in the assay, whether cell migration in the presence of the LMWH is within a predetermined second range, which is lower than the first range. If the LMWH is within the predetermined second range, it will qualify as M402 or an M402 precursor.

The assay includes a first part that is a cell culture in an assay medium, a second part that is chamber, and a third part that is a stimulant. The chamber comprises at least a first and a second compartment separated by a porous membrane. The cells are placed in the first compartment of the chamber, while the stimulant and LMWH sample are placed in the second compartment of the chamber, allowing assessment of the activity of the LMWH by measuring the level of cell migration from the first to the second compartment of the chamber. The activity of the LMWH refers to the biological activity of the LMWH (e.g., potency of the LMWH).

A sample of a batch of LMWH qualifies as M402 (e.g., qualifies for use as M402 or qualifies for subsequent processing into M402) if the activity of the sample assessed using the methods disclosed herein meets (e.g., corresponds with, satisfies, or falls within) a predetermined range (e.g., a range including the minimum and maximum values of the range, and in some cases plus or minus a window of variability (e.g., +/−0.5%, +/−1%, +/−5%, or +/−10%); or value (e.g., an average value (or other value of central tendency) plus or minus a window of variability (e.g., +/−0.5%, +/−1%, +/−5%, or +/−10%)).

For example, a sample of a batch of LMWH qualifies as M402 or an M402 precursor if the activity of the sample assessed using the methods disclosed herein corresponds to the activity of a validated sample of M402 under the same conditions and at the same concentration. In some instances, the activity of the sample meets the activity of the validated sample if the activity of the sample is within 70%-130% (e.g., 80%-125%, 80%-120%, 90%-110%, or 95%-105%) of the activity of the validated sample at the same concentration and under the same experimental conditions. Alternatively, a sample of a batch of LMWH can qualify as M402 or an M402 precursor if the activity of the sample assessed using the methods disclosed herein inhibits cell migration in the potency assay within a predetermined range which is less than a predetermined range of a reference value (e.g., cell migration in response to a stimulant (e.g., SDF1α) in the absence of the LMWH).

In some embodiments, the activity of the LMWH is expressed as a single value (e.g., an average value (or other value of central tendency) plus or minus a window of variability (e.g., +/−0.5%, +/−1%, +/−5%, or +/−10%)) and the predetermined activity of the validated sample is also expressed as a single value (e.g., an average value (or other value of central tendency) plus or minus a window of variability (e.g., +/−0.5%, +/−1%, +/−5%, or +/−10%)). For example, a sample of a batch of LMWH can qualify as M402 or an M402 precursor if the activity of the sample assessed using the methods disclosed herein and expressed as a single value (e.g., an average value (or other value of central tendency) plus or minus a window of variability (e.g., +/−0.5%, +/−1%, +/−5%, or +/−10%)) falls within a certain percentage of (e.g., within 70%-130%) of the predetermined value.

In some embodiments, the activity of the LMWH is expressed as a single value (e.g., an average value (or other value of central tendency) plus or minus a window of variability (e.g., +/−0.5%, +/−1%, +/−5%, or +/−10%)) and the activity of the validated sample is expressed as a range (e.g., a range including the minimum and maximum values of the range, and in some cases plus or minus a window of variability (e.g., +/−0.5%, +/−1%, +/−5%, or +/−10%)). For example, a sample of a batch of LMWH can qualify as M402 or an M402 precursor if the activity of the sample assessed using the methods disclosed herein and expressed as an average value plus or minus a window of variability (e.g., +/−0.5%, +/−1%, +/−5%, or +/−10%) falls within a predetermined range (e.g., a range including the minimum and maximum values of the range, and in some cases plus or minus a window of variability (e.g., +/−0.5%, +/−1%, +/−5%, or +/−10%)).

The predetermined value or range as described herein and/or the value or range expressing the activity of the LMWH can be recorded, e.g., using a recordable medium (e.g., on paper, in a computer readable medium, or in an ELN file, e.g., in a Certificate of Testing, Certificate of Analysis, Material Safety Data Sheet (MSDS), batch record, or Certificate of Analysis (CofA)).

### Assays

Assays described herein can include a cell based migration assay or kit. For example, the assay can measure the migration of cells in the presence and absence of a LMWH in response to a stimulant which known to induce cell migration in the absence of the LMWH and which cell migration is inhibited by the LMWH. The assays described herein are quantitative, reproducible, accurate, and precise.

Cell Migration Assay Kit

Detection of cell migration, as described herein, can utilize any suitable method known in the art or described herein. Conventional transchamber/transwell migration assays/kits can be used. An exemplary transchamber cell migration assay plate is described below.

An exemplary transchamber plate may contain a hollow first chamber, sealed at one end with a porous membrane separating the first chamber from a second chamber. Cells are initially seeded in the first chamber; while a compound of interest (e.g., a LMWH, e.g., M402) and/or a stimulant, e.g., a protein, (e.g., SDF1α) are contained in the media of the second chamber. The cells are then incubated for a period of time sufficient to allow migration of the cells through the pores of the membrane and into the second chamber. Migratory cells in the second chamber can then be stained and counted using any suitable method known in the art, e.g., fluorescent staining, and analysis by, e.g., a fluorescent plate reader.

The components of the assay can be determined by one of skill in the art, exemplified components are described herein:

Chamber

Conventional transchamber migration kits and cell culture inserts are known in the art and commercially available (e.g., Life Technologies, Corning Inc. (e.g., catalog #3387 or 3388), Becton Dickinson, Merck, and Millipore). Transwell assays for high throughput screening of migration are also available and known in the art (e.g., 96 cell format (e.g., disposable ChemoTx system from Neuro Probe).

Cell migration assays described herein can be carried out using any suitable cell culture chamber known in the art (e.g., Boyden chambers, bridge chambers, Dunn chambers; multiwell chambers; capillary chambers). An exemplary method of analyzing cell migration is a Boyden chamber. Other exemplary migration assays known in the art include, but are not limited to, the cell exclusion zone assay; fence assay (ring assay); microcarrier bead assay; spheroid migration assay; capillary chamber migration assay (microfluidic chamber assays); scratch assay; capillary tube migration assays (e.g., leukocyte migration agarose technique assay; single cell motility assay (e.g., colloidal particle assay; colloidal gold single cell); time lapse/cell tracking; agar plate techniques; T maze technique, aggregation techniques; filter immersion techniques; opalescency techniques (Kramer N, et al., Mutation Research 752: 10-24 (2013)).

Cells

A suitable cell line can be determined by one of skill in the art. Exemplary cell lines include but are not limited to Jurkat (e.g., Jurkat Clone E6-1 (ATCC, TIB-152)), CHO, HEK293, C127, VERO, BHK, HeLa, COS, MDCK, etc (as referenced in U.S. Pat. No. 6,632,637, the contents of which are incorporated by reference).

Membrane

The membrane can be made of any suitable medium known in the art, e.g., polycarbonate, polyethylene terephthalate (PET). The pore size of the membrane can depend on the cell line employed. A suitable pore size can be determined by one of ordinary skill in the art. By way of exemplification, in a standard Boyden assay, the pore diameter is typically 3 to 12 μm, and is selected to suit the subject cells. Smaller pore sizes result in a greater challenge for the migrating cell to pass through the membrane. Most cells range in size from 30-50 μm and can migrate efficiently through 3-12 μm pores, whereas, lymphocytes (10 μm) can migrate through pores as small as 0.3 μm. The pore size can be, but is not limited to 1 μm, 2 μm, 3 μm, 4 μm, 5 μm, 6 μm, 7 μm, 8 μm, μm 9 μm, 10 μm, 11 μm, or 12 μm; between 1-12 μm; between 3-8 μm, or between 5-12 μm; greater than 0.3 μm, 1 μm, 5 μm, 10 μm, or 12 μm. In some embodiments, the pore size is 5 μm. The medium used in the top chamber can be serum free.

Compound of Interest

The compound of interest (e.g., the LMWH, e.g., M402) can be utilized at any suitable concentration determined by the user. The concentration of the compound can depend on the cells, compounds of interest (e.g., M402), and the stimulant, e.g., protein (e.g., SDF1α) being employed. Multiple concentrations of the compound of interest can be utilized, e.g., such as serial dilutions (e.g., serial dilutions of the M402 sample resulting in final M402 concentrations of about 4100 μg/ml; 1782.6 μg/ml; 775 μg/ml; 337 μg/ml; 146.5 μg/ml; 63.7 μg/ml; 27.7 μg/ml; 12 μg/ml; 5.2 μg/ml; and 2.3 μg/ml). Exemplary concentrations of the compound (e.g., M402) also include, but are not limited to, from 1 to 10,000 μg/ml, 1 to 9000 μg/ml, 1 to 8000 μg/ml, 1 to 7000 μg/ml, 1 to 6000 μg/ml, 1 to 5000 μg/ml, 1 to 4000 μg/ml, 1 to 4500 μg/ml, and additionally any combination of one or one or more of about 1 μg/ml, about 2 μg/ml, about 2.3 μg/ml, about 2.5 μg/ml, about 5 μg/ml, about 5.2 μg/ml, about 10 μg/ml, about 12 μg/ml, about 15 μg/ml, about 20 μg/ml, about 25 μg/ml, about 27 μg/ml, about 50 μg/ml, about 60 μg/ml, about 63 μg/ml, about 75 μg/ml, about 100 μg/ml, about 140 μg/ml, about 146 μg/ml, about 150 μg/ml, about 200 μg/ml, about 250 μg/ml, about 300 μg/ml, about 337 μg/ml, about 350 μg/ml, about 400 μg/ml, about 450 μg/ml, about 500 μg/ml, about 550 μg/ml, about 600 μg/ml, about 650 μg/ml, about 700 μg/ml, about 750 μg/ml, about 775 μg/ml, about 800 μg/ml, about 900 μg/ml, about 1000 μg/ml, about 1100 μg/ml, about 1500 μg/ml, about 1700 μg/ml, about 1782 μg/ml, about 2000 μg/ml, about 3000 μg/ml, about 4000 μg/ml, about 5000 μg/ml, about 6000 μg/ml, about 7000 μg/ml, about 8000 μg/ml, about 9000 μg/ml, and about 10000 μg/ml.

Stimulant

The stimulant, e.g., protein (e.g., SDF1α) can be utilized at any suitable concentration determined by the user. The concentration of the stimulant can depend on the compound, cells, and/or stimulant, e.g., protein (e.g., SDF1α) being employed. Exemplary concentrations of the compound include but are not limited to, from 1 to 100 ng/ml, 1 to 50 ng/ml, 1 to 25 ng/ml, 1 to 10 ng/ml, about 1 ng/ml, about 2 ng/ml, about 5 ng/ml, about 10 ng/ml, about 25 ng/ml, about 50 ng/ml, about 100 ng/ml, at least 0.5 ng/ml, at least 1 ng/ml, at least 5 ng/ml, at least 10 ng/ml, at least 25 ng/ml, at least 50 ng/ml, or at least 100 ng/ml.

Cell Culture Media

Any suitable cell culture media can be used. The cell culture media can depend on the cell line selected, e.g., any cell culture media suitable for the culture of the selected cell line. One of ordinary skill in the art can determine a suitable cell culture medium. In some embodiments, the cell culture media is Hanks. Suitable cell culture media can include, but is not limited to, Hanks, OptiMEM, RPMI, DMEM, MEM, Hams, EMEM, and F-12. The cell culture medium can be serum free.

The individual parameters of the assay can be determined by one of skill in the art, exemplary parameters are defined herein:

Incubation Periods

Post seeding of the cells in the first chamber and addition of the compound (e.g., LMWH, e.g., M402) and/or the stimulant, e.g., the protein (e.g., SDF1α) to the second chamber, the cells can be incubated for any suitable period of time known in the art and/or determined by the user based on the cells, compound of interest (e.g., M402), and/or stimulant, e.g., protein (e.g., SDF1α) being employed, to allow for cell migration. Exemplary incubation times include, but are not limited to, at least 1 hour (e.g., at least 2 hours, at least 4 hours, at least 6 hours, at least 9 hours, at least 12 hours, at least 18 hours, at least 24 hours, from 1 to 24 hours, from 1 to 12 hours, from 1 to 6 hours, from 1 to 4 hours, from 2.5 to 12 hours, from 2.5 to 4 hours, from 6 to 12 hours, 1 hour, 2 hours, 4 hours, 6 hours, 8 hours, 10 hours, 12 hours, 28 hours, 24 hours, more than 1 hour, more than 2 hours, more than 4 hours, more than 6 hours, more than 8 hours, more than 10 hours, more than 12 hours, more than 18 hours, more than 24 hours. Incubation can be carried out at any suitable temperature known in the art or determined by the user based on the cells, compounds of interest (e.g., M402), and stimulant, e.g., protein (e.g., SDF1α) being employed. Exemplary incubation temperatures include, but are not limited to, around 37° C., at least 25° C., at least 37° C., between 25-40° C. Exemplary incubation CO₂ levels, include, but are not limited to 5%.

Cell Density

Cells can be seeded in the first chamber at any suitable concentration known in the art or determined by the user. The determination can be based on the size of the well, the cells being used, compound of interest (e.g., M402), and the stimulant, e.g., protein (e.g., SDF1α) being employed. Exemplary cell concentrations, include, but are not limited to, from 2000 to 2 million cells per well, about 0.3 million cells per well, about 0.5 million cells per well, about 1 million cells per well, about 1.5 million cells per well, or about 2 million cells per well.

Pre-Incubation of the Compound of Interest and Stimulant

The compound of interest (e.g., the LMWH, e.g., M402) and the stimulant, e.g., the protein (e.g., SDF1α) can be pre-incubated together prior to addition to the chamber. The pre-incubation can be carried out for any suitable incubation period determined by one of skill in the art. Exemplary incubation periods can include, but are not limited to, from 30 minutes to 12 hours, from 30 minutes to 6 hours, from 30 minutes to 3 hours, at least 30 minutes, at least 30 minutes, at least 45 minutes, at least 1 hour, at least 3 hours, at least 6 hours, at least 9 hours, at least 12 hours, about 15 minutes, about 30 minutes, about 45 minutes, about 1 hour, about 3 hours, about 6 hours, or about 12 hours. The incubation can be carried out at any suitable temperature as determined by the user. Exemplary incubation temperatures can include, but are not limited to, room temperature, about 25° C., about 37° C., at least room temperature, at least 25° C., at least 37° C.

### SDF-1α/CXCL12

Stromal cell derived factor 1 (SDF-1α) (also known as CXCL12; chemokine (C-X-C Motif) ligand 12; chemokine CXC ligand 12; SDF1A, SDF1B, SDF1, intercrine reduced in hapatomas, pre-B cell growth stimulating factor; IRH; PBSF; SCYB12; TLSF, TPAR1; and SDF-1) is a member of the intercrine protein family. SDF-1α functions as a ligand for the G-protein coupled receptor, chemokine (C-X-C motif) receptor 4 (CXCR4). Activation of the CXCR4 receptor induces a rapid and transient rise in the level of intracellular calcium ions and chemotaxis. SDF-1α also binds CXCR7, which activates the beta-arrestin pathway and acts as a scavenger receptor for SDF-1. SDF-1α plays a role in many diverse cellular functions, including embryogenesis, immune surveillance, inflammatory response, tissue homeostasis, and tumor growth and metastasis.

The SDF-1α amino acid and nucleotide sequences are known in the art. SDF-1α is known in the art to have multiple isoforms. An exemplary amino acid and nucleotide sequence for human SDF-1α are provided herein as SEQ ID NO: 1 and SEQ ID NO: 2, respectively.

| NCBI Reference Sequence: NP_954637.1 | |
|---|---|
| SEQ ID NO: 1 | |
| 1 | mnakvvvvlv lvltalclsd gkpvslsyrc pcrffeshva ranvkhlkil ntpncalqiv |
| 61 | arlknnnrqv cidpklkwiq eylekalnk. |
| NCBI Reference Sequence: NM_199168.3 | |
| SEQ ID NO: 2 | |
| 1 | gccgcacttt cactctccgt cagccgcatt gcccgctcgg cgtccggccc ccgacccgcg |
| 61 | ctcgtccgcc cgcccgcccg cccgcccgcg ccatgaacgc caaggtcgtg gtcgtgctgg |
| 121 | tcctcgtgct gaccgcgctc tgcctcagcg acgggaagcc cgtcagcctg agctacagat |
| 181 | gcccatgccg attcttcgaa agccatgttg ccagagccaa cgtcaagcat ctcaaaattc |
| 241 | tcaacactcc aaactgtgcc cttcagattg tagcccggct gaagaacaac aacagacaag |
| 301 | tgtgcattga cccgaagcta aagtggattc aggagtacct ggagaaagct ttaaacaagt |
| 361 | aagcacaaca gccaaaaagg actttccgct agacccactc gaggaaaact aaaaccttgt |
| 421 | gagagatgaa agggcaaaga cgtgggggag ggggccttaa ccatgaggac caggtgtgtg |
| 481 | tgtggggtgg gcacattgat ctgggatcgg gcctgaggtt tgccagcatt tagaccctgc |
| 541 | atttatagca tacggtatga tattgcagct tatattcatc catgccctgt acctgtgcac |
| 601 | gttggaactt ttattactgg ggtttttcta agaaagaaat tgtattatca acagcatttt |
| 661 | caagcagtta gttccttcat gatcatcaca atcatcatca ttctcattct cattttttaa |
| 721 | atcaacgagt acttcaagat ctgaatttgg cttgtttgga gcatctcctc tgctcccctg |
| 781 | gggagtctgg gcacagtcag gtggtggctt aacagggagc tggaaaaagt gtcctttctt |
| 841 | cagacactga ggctcccgca gcagcgcccc tcccaagagg aaggcctctg tggcactcag |
| 901 | ataccgactg gggctgggcg ccgccactgc cttcacctcc tctttcaacc tcagtgattg |
| 961 | gctctgtggg ctccatgtag aagccactat tactgggact gtgctcagag acccctctcc |
| 1021 | cagctattcc tactctctcc ccgactccga gagcatgctt aatcttgctt ctgcttctca |
| 1081 | tttctgtagc ctgatcagcg ccgcaccagc cgggaagagg gtgattgctg gggctcgtgc |
| 1141 | cctgcatccc tctcctccca gggcctgccc cacagctcgg gccctctgtg agatccgtct |
| 1201 | ttggcctcct ccagaatgga gctggccctc tcctggggat gtgtaatggt ccccctgctt |
| 1261 | acccgcaaaa gacaagtctt tacagaatca aatgcaattt taaatctgag agctcgcttt |
| 1321 | gagtgactgg gttttgtgat tgcctctgaa gcctatgtat gccatggagg cactaacaaa |
| 1381 | ctctgaggtt tccgaaatca gaagcgaaaa aatcagtgaa taaaccatca tcttgccact |
| 1441 | accccctcct gaagccacag cagggtttca ggttccaatc agaactgttg gcaaggtgac |
| 1501 | atttccatgc ataaatgcga tccacagaag gtcctggtgg tatttgtaac tttttgcaag |
| 1561 | gcattttttt atatatattt ttgtgcacat ttttttttac gtttctttag aaaacaaatg |
| 1621 | tatttcaaaa tatatttata gtcgaacaat tcatatattt gaagtggagc catatgaatg |
| 1681 | tcagtagttt atacttctct attatctcaa actactggca atttgtaaag aaatatatat |
| 1741 | gatatataaa tgtgattgca gcttttcaat gttagccaca gtgtattttt tcacttgtac |
| 1801 | taaaattgta tcaaatgtga cattatatgc actagcaata aaatgctaat tgtttcatgg |
| 1861 | tataaacgtc ctactgtatg tgggaattta tttacctgaa ataaaattca ttagttgtta |
| 1921 | gtgatggagc ttaaaaaaaa. |

In some embodiments, SDF-1α is recombinant SDF-1α. In some embodiments, SDF-1α is human recombinant SDF-1α. In some embodiments, SDF-1α is wild type human recombinant SDF-1α. In some embodiments, SDF-1α is any isoform of human SDF-1α known in the art. In some embodiments, SDF-1α or fragment thereof comprises, consists essentially of, or consists of: an amino acid sequence which is at least 60, 70, 80, 90, 95, 99 or 100% percent identical to the amino acid sequence of SEQ ID NO: 1; an amino acid sequence having at least one, two, three, five, ten, fifteen but not more than thirty, twenty modifications, e.g., substitutions, of an amino acid sequence of SEQ ID NO:1; an amino acid sequence of at least 20, 25, 30, or 40 contiguous amino acids of SEQ ID NO: 1 or an amino acid sequence of SEQ ID NO: 1.

### LMWH

In some instances, the LMWH is described in U.S. Pat. No. 8,592,393; U.S. Pat. No. 8,569,262; U.S. Pat. No. 7,790,700; U.S. Pat. No. 7,781,416; U.S. Pat. No. 8,222,231; U.S. Pat. No. 8,067,555; U.S. Pat. No. 5,767,269; U.S. Pat. No. 5,763,427; U.S. Pat. No. 5,744,457; U.S. Pat. No. 6,001,820; U.S. Pat. No. 6,498,246; U.S. Pat. No. 8,018,231; U.S. Pat. No. 5,280,016; U.S. Pat. No. 5,696,100; U.S. Pat. No. 5,296,471; U.S. Pat. No. 5,707,974; U.S. Pat. No. 5,668,118; U.S. Pat. No. 5,990,097; U.S. Pat. No. 5,795,875; U.S. Pat. No. 5,605,891; U.S. Pat. No. 5,206,223; U.S. Pat. No. 7,285,536; U.S. Pat. No. 8,209,131; U.S. Pat. No. 4,990,502; U.S. Pat. No. 7,468,358; U.S. Pat. No. 7,230,096; U.S. Pat. No. 6,329,351; U.S. Pat. No. 7,005,508; U.S. Pat. No. 5,541,166; US20100249061; US20080254487; US20080227752; US20070021378; US20130323230; US20120196828; US20090054374; US20070123489; US20060040896; and US20050282775; the contents of each of which are incorporated by reference.

In some embodiments, the LMWH preparation comprises a polysaccharide of Formula (I)

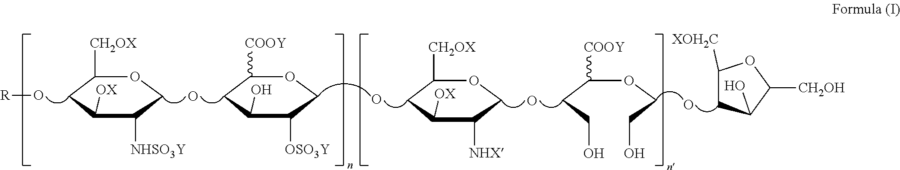

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

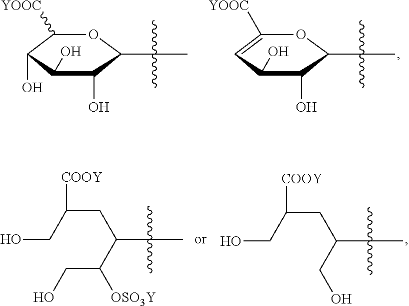

each Y is independently a singularly charged cation such as Na⁺, K⁺, or NH₄⁺.

In some embodiments, the polysaccharide of Formula (I) is a polysaccharide of Formula (Ia)

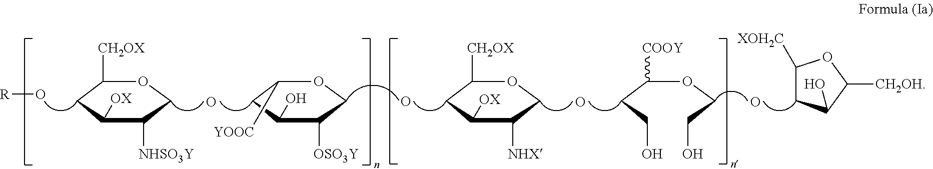

In some embodiments, Y for each occurrence is Na⁺.

In some embodiments, R is,

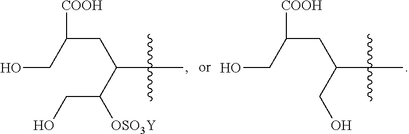

In some embodiments, the polysaccharide of Formula (I) is a polysaccharide of Formula (Ib)

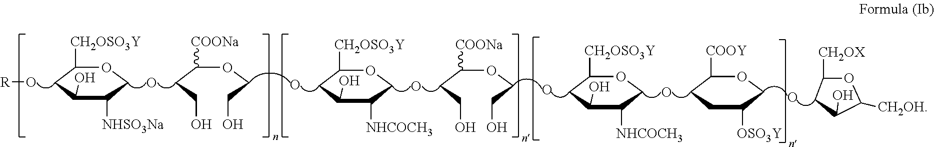

In some embodiments, Y for each occurrence is Na⁺.

In some embodiments, R is,

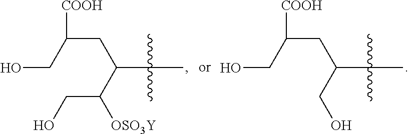

In some embodiments, the preparation consists essentially of polysaccharides having the structure of Formula (I) or Formula (Ia) or Formula (Ib). In some embodiments, the preparation consists of polysaccharides having the structure of Formula (I) or Formula (Ia) or Formula (Ib).

In some embodiments, at least about 20% (e.g., at least about 30%, at least about 40%, at least about 50%, at least about 60%, at least about 70%, at least about 80%, at least about 90%, at least about 95%, at least about 98%, or at least about 99%) of the polysaccharides in the preparation have the structure of Formula (I) or Formula (Ia).

### M402

“M402” as used herein refers to a LMWH having one or more of the following characteristics: (a) a weight average chain molecular weight between 3,500 and 8,000 Da; (b) anti-Xa activity of less than 20 IU/mg and anti-IIa activity of 20 IU/mg or less; (c) greater than 5% and less than 25% glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some instances the LMWH comprises polysaccharides that comprise Formula I:

[U_{w}—H_{x,y,z}]ₘ˜[U_{G}—H_{x,y,z}]ₙ

wherein each occurrence of U indicates a uronic acid residue and each occurrence of H indicates a hexosamine residue;

wherein m and n are integers such that

m=4-16, and

n=1-4;

each of w, x, y and z can, independently, be the same or different for each occurrence of [U_{w}—H_{x,y,z}] and each of x, y and z can, independently, be the same or different for each occurrence of [U_{G}—H_{x,y,z}], wherein

w=−2OS or −2OH;

x=—NS or —NAc;

y=−3OS or −3OH;

z=−6OS or −6OH;

and

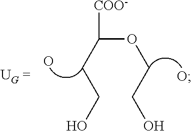

wherein the symbol ˜ indicates that the units marked m and n are distributed along the polysaccharide chain and are not necessarily in sequence. In some instances, n=1-3.

In some instances, the M402 preparation has an anti-Xa activity of less than 15 IU/mg. In some instances, the M402 preparation has an anti-Xa activity of less than 10 IU/mg. In some instances, the M402 preparation has an anti-IIa activity of less than 1 IU/mg.

In some instances, the reducing end further comprises a 2,5-anhydromannitol residue. In some instances, about 50% of the reducing ends comprise a 2,5-anhydromannitol residue. In some instances, the reducing end further comprises a 2,5-anhydromannitol residue. In some instances, the polysaccharides of the M402 preparation have a uronic acid at the non-reducing end. In some instances, the polysaccharides of the M402 preparation have a non native uronic acid at the non-reducing end. In some instances, the polysaccharides of the M402 preparation have a glycol split uronic acid at the non-reducing end.

In some instances, M402 has greater than 5% and less than 20% glycol split uronic acid residues; or between 10% and 20% glycol split uronic acid residues. In some instances, M402 has one or more chains having a glycol split uronic acid residue and polysaccharide chains of the preparation each having no more than 3 glycol split uronic acid residues (U_{G}).

In some instances, each polysaccharide chain of the M402 preparation has no more than 2 glycol split uronic acid residues (U_{G}). In some instances, each polysaccharide chain of the M402 preparation ha greater than 40% U_{2S}H_{NS,6S} (e.g., greater than, 70%, U₂ₛH_{NS,6S}) disaccharide residues. In some instances, M402 has a degree of desulfation less than 40% (e.g., less than 30%, less than 10%.).

In some instances, the M402 preparation has a polydispersity of about 1.2 to 1.7. In some instances, M402 has one or more of a sodium content less than 30%; less than 20 ppm iodine; less than 30% sulfur; and less than 50 ppm boron.

### Methods of Manufacturing LMWH Preparations and Compositions

A LMWH preparation can be made, e.g., by known methods, e.g., see U.S. Pat. No. 8,592,393; U.S. Pat. No. 8,569,262, the contents of each of which are incorporated by reference. In some embodiments, a LMWH preparation lacking substantial anticoagulant activity can be made by a method that includes providing a precursor LMWH preparation (e.g., unfractionated heparin (UFH), having a weight average molecular weight of greater than 7000 Da or a chain length of greater than 7 to 18 disaccharides, and processing the precursor LMWH preparation (e.g., by enzymatic or chemical depolymerization, e.g., by nitrous acid depolymerization) to obtain a LMWH preparation having a weight average molecular weight of about 3000 to 8000 Da or an average chain length of about 7 to 16 disaccharides. The LMWH preparation can be further oxidized using periodate (e.g., treatment with 0.05 M to 0.2 M sodium periodate for about 10 to 20 hours at 0 to 10° C.). The oxidized preparation can then be further reduced by treatment with a reducing agent (e.g., treatment with sodium borohydride for 0.5 to 2.0% (w/v)) for 0.5 to 3 hours at a pH of about 6.0 to 7.0 at 0 to 10° C.), to produce a LMWH preparation lacking substantial anticoagulant activity. A sample of the LMWH can be obtained and assessed using methods disclosed herein, to determine if the LMWH qualifies for use as M402 or for subsequent processing into M402. If the sample qualifies for subsequent processing into M402, the LMWH preparation (e.g., drug substance) can be further processed, (e.g., formulated into a LMWH composition (e.g., drug product)).

### Pharmaceutical Compositions

Pharmaceutical compositions described herein can include an aqueous formulation prepared including the therapeutic agent in a pH-buffered solution. A “buffer” as used herein is an agent that maintains a stable pH in a solution within a specific pH range. Buffering ranges are determined by pKa. In some embodiments, a composition described herein includes a buffering agent. Examples of potential buffering agents include a citrate buffer, a phosphate buffer, a histidine buffer, a maleate buffer, a succinate buffer, an acetate buffer, a malate buffer, or any combination thereof.

In some embodiments, a composition described herein includes a LMWH preparation formulated at a pH, e.g., a buffered pH, which maintains the stability of the polysaccharide chains. As used herein, “pH” or “pKa” units or values can include +/−0.5 (e.g., 0.1, 0.2, 0.3, 0.4, 0.5, 1.0, 2.0, 2.5) units. As used herein, “pH” or “pKa” units or values can include +/−0.5 (e.g., 0.1, 0.2, 0.3, 0.4, 0.5, 1.0, 2.0, 2.5) units at a predetermined temperature, e.g., 0-50° C. In some embodiments, the composition is formulated at a pH, e.g., a buffered pH, that results in a composition that is substantially free of formic acid and/or C₂H₆O₃. pH can be determined using standard methods, e.g., methods described herein.

In some embodiments, a composition described herein includes a LMWH preparation formulated at a pH, e.g., a buffered pH, which minimizes degradation of the polysaccharide chains at a predefined temperature, e.g., at about 2-8° c., e.g., 4° C.

In some embodiments, a composition described herein includes a LMWH preparation formulated at a pH, e.g., a buffered pH, which minimizes degradation of the LMWH preparation at a predefined temperature, for a predetermined time, e.g., days, weeks, months, years. In some embodiments, a composition described herein includes a LMWH preparation formulated at a pH, e.g., a buffered pH, which minimizes degradation of the LMWH preparation at a predefined temperature, for a predetermined time, e.g., 1-3 months, 3-6 months, 6-9 months, 9-12 months, 1-12 months, 1-24 months, 1-36 months, 1-6 months, 1-9 months. In some embodiments, a composition described herein includes a LMWH preparation formulated at a pH, e.g., a buffered pH, which minimizes degradation of the LMWH preparation at a predefined temperature, for a predetermined time, e.g., up to 3 months, up to 6 months, up to 9 months, up to 12 months, up to 24 months, up to 36 months. In some embodiments, a composition described herein includes a LMWH preparation formulated at a pH, e.g., a buffered pH, which minimizes degradation of the LMWH preparation at a predefined temperature, for a predetermined time, e.g., at least 3 months, at least 6 months, at least 9 months, at least 12 months, at least 24 months, at least 36 months.

In some embodiments, a composition described herein has a pH between about 4.0-9.0 or above. In some embodiments, the composition has a pH between about 5.0-9.0, 5.0-8.0, 5.5-8.0, 6.0-8.0, 6.1-8.0, 6.2-8.0, 6.3-8.0, 6.4-8.0, or 6.5-8.0. In some embodiments, the composition has a pH of about 6.0 or above, 6.1 or above, 6.2 or above, 6.3 or above, 6.4 or above, or 6.5 or above. In some embodiments, the composition has a pH of about 6.2. In some embodiments, the composition has a pH of about 5.0 or above, 6.0 or above, 7.0 or above, 8.0 or above, 9.0 or above, or 10.0 or above.

In some embodiments, a composition described herein has a pH of about 8.0. In some embodiments, a composition described herein has a pH of about 7.5. In some embodiments, a composition described herein has a pH of about 7.6. In some embodiments, a composition described herein has a pH of about 7.7. In some embodiments, a composition described herein has a pH of about 7.8. In some embodiments, a composition described herein has a pH of about 7.9. In some embodiments, a composition described herein has a pH of about 8.1. In some embodiments, a composition described herein has a pH of about 8.2. In some embodiments, a composition described herein has a pH of about 8.3. In some embodiments, a composition described herein has a pH of about 8.4. In some embodiments, a composition described herein has a pH of about 8.5.

### Articles of Manufacture

The methods described herein can further include processing a LMWH into an article of manufacture which, optionally, provides instructions for its use. The article of manufacture can comprise a container. Suitable containers include, for example, bottles, vials (e.g., single chamber vial, or dual chamber vials), syringes (e.g., a single chamber syringe, or a dual chamber syringes) and test tubes. The container may be formed from a variety of materials such as glass or plastic. The container holds the composition and the label on, or associated with, the container may indicate directions for use. For example, the label may further indicate that the composition is useful or intended for subcutaneous or intravenous administration. The container holding the composition may be a multi-use vial, which allows for repeat administrations (e.g. from 2-6 administrations) of the composition. The article of manufacture can also include other materials desirable from a commercial and user standpoint, including other buffers, diluents, filters, needles, syringes, and package inserts with instructions for use.

In some embodiments, an article of manufacture described herein, can include an anaerobic overlay, e.g., a nitrogen overlay, over the composition. The anaerobic overlay can or cannot include CO₂. In some aspects, the disclosure features an article of manufacture that contains a composition described herein. In some embodiments, the article of manufacture can include a container, e.g., a container described herein, that includes a pharmaceutical composition described herein. In some embodiments, the container can further include an anaerobic overlay, e.g., a nitrogen overlay. In some embodiments, anaerobic (or minimal oxygen) conditions can be used to maintain the stability of the glycol split LMWH, e.g., under low certain pH conditions (e.g., low pH conditions, e.g., less than pH 6.0). In some instances, anaerobic conditions can be created by replacing air in the headspace of a vial containing liquid M402. Exemplary agents that can be used to replace air include, but are not limited to, nitrogen, an inert gas, e.g., argon. In some embodiments, an anaerobic overlay does not include CO₂. In some embodiments, an anaerobic overlay can include CO₂. In some instances, Nitrogen (N2) can be used to assess the impact of headspace gas on stability of the LMWH preparation. The N2 overlay can for example, be prepared by placing empty glass vials in a box with an N2 environment (99.99% purity N2), purging air from the vials with N2, and adding prepared preparations to the vials. In some embodiments, a glove box can be purged with N2 gas, maintaining positive pressure and a % RH of less than 10%. For each preparation, empty vials can be placed in the glove box, and using a secondary N2 source with tubing, N2 dispensed into each vial to displace air by holding the tubing over each vial for 4-5 seconds.

### Other Embodiments

This invention is further illustrated by the following examples that should not be construed as limiting. The contents of all references, patents and published patent applications cited throughout this application are incorporated herein by reference.

### EXAMPLES

### Example 1

### Assessment of M402 Using Cell Based Assay

A qualified sample of M402 was exposed to an assay that includes a population of cells responsive to SDF1α described herein. As described herein, the assay included a Boyden chamber to allow analysis of cell migration in response to a constant concentration of the cytokine SDF1α in the absence and presence of different doses of M402. Jurkat cells were seeded in the first chamber (about 300,000 cells per well in Hanks serum free medium). SDF1α (4 ng/ml) and M402 (2.3 μg/ml-4100 μg/ml) were pre-incubated together for 45±10 minutes at room temperature; added to the second chamber; and incubated for 3.5 hours to allow migration. Cells in the second chamber were subsequently collected via pipette extraction; and quantified using the metabolic dye AlamarBlue. The activity of the cells in the presence of M402 was recorded for use as a reference value/standard for qualifying LMWH as M402.

### Example 2

### Manufacture of LMWHs Utilizing Cell Based Assay

The following Example describes methods of manufacturing M402 comprising qualifying a LMWH as M402.

A batch of LMWH (Batch A) was manufactured by depolymerizing an unfractionated heparin (UFH) with about 0.02 to 0.04 M nitrous acid (HONO) at a pH of about 2 to 4 for about 1 to 5 hours at a temperature of about 10 to 30° C. to yield a depolymerized polysaccharide preparation; oxidizing the depolymerized polysaccharide preparation with periodate; reducing the oxidized polysaccharide preparation with sodium borohydride; and isolating the reduced LMWH. A sample of the batch of LMWH (Sample A) was taken to determine whether or not the batch qualified as M402 using the assay described in Example 1, which included assessing whether the activity of the LMWH was within a predetermined range that qualified the LMWH as M402.

The assay was performed as described in Example 1 but substituting M402 for the sample of the batch of LMWH. The assay was repeated for a second sample of Batch A, Sample B. The assay was also performed for various control samples that were expected to not qualify as M402. Those control samples include: Sample C, in which the LMWH was diluted 50% in water, Sample D, in which the LMWH of Batch A was subjected to forced degradation conditions; enoxaparin (a distinct and commercially available LMWH); M202 (a distinct, 100% N-desulfated LMWH); UFH SPL (an unfractionated full length heparin); and an M402 manufacturing process ‘Intermediate’.

The activity of the samples was reported as a relative percentage of the standard (see Example 1), as calculated by parallel line analysis (PLA) known to one of skill in the art (see Table 1).

TABLE 1
Specificity of Cell Based Migration Assay

| | % Migration of | |
|---|---|---|
| | Qualified M402 | |
| Sample | Reference Standard | Qualify as M402 |
| Batch A, Sample A | 91.6 | ✓ |
| Batch A, Sample B | 97.450 | ✓ |
| Batch A, Sample C | 0.6% | X |
| (Diluted 50%) | | |
| Batch A, Sample D | 68.6 | X |
| (Forced Degradation) | | |
| Enoxaparin | 25.6% | X |
| M202 | 1.9% | X |
| UFH SPL | 130.5 | X |
| Intermediate | 62.7 | X |

As shown in Table 1, Samples A and B, from Batch A, qualified as M402, whereas Samples C, D, enoxaparin, M202, UFH SPL, and intermediate do not qualify as M402.

1. A method of manufacturing a LMWH (e.g., an M402) pharmaceutical composition, the method comprising:
producing a batch of a LMWH preparation (e.g., a drug substance);
determining for a sample of the batch whether the activity of the LMWH preparation is within a predetermined range by:
exposing the sample to an assay comprising a population of cells that respond to a stimulant (e.g., a protein, e.g., a cytokine, e.g., SDF1α) in the absence of the LMWH within a predetermined first range; and
determining, in the assay, whether the cellular response in the presence of the LMWH is within a predetermined second range (e.g., which is lower than said first range); and
formulating the batch of LMWH (e.g., for medical use), to thereby manufacture a LMWH (e.g., an M402) pharmaceutical composition (e.g., a drug product).

2-67. (canceled)
